(19) United States
(12) Patent Application Publication
(10) Pub. No.: US 2018/0318603 A1
(43) Pub. Date: Nov. 8, 2018

(54) RADIATION MODULATOR AND METHODS OF USE AND PRODUCTION THEREOF

(71) Applicants: Chunjoo Park, St. Louis, MO (US); Sasa Mutic, St. Louis, MO (US); Hao Zhang, St. Louis, MO (US)

(72) Inventors: Chunjoo Park, St. Louis, MO (US); Sasa Mutic, St. Louis, MO (US); Hao Zhang, St. Louis, MO (US)

(73) Assignee: Washington University, St. Louis, MO (US)

(21) Appl. No.: 15/972,907

(22) Filed: May 7, 2018

Related U.S. Application Data
(60) Provisional application No. 62/502,092, filed on May 5, 2017.

(51) Int. Cl. A61N 5/10

(52) U.S. Cl. CPC A61N 5/1016; A61N 5/1039; A61N 2005/1003; A61N 5/1007; A61N 5/1027

(57) ABSTRACT

The present disclosure is directed to a computer-implemented method for designing a patient-specific brachytherapy (BT) tandem applicator. The method is implemented using at least one processor in communication with at least one memory. The method includes receiving a radiation treatment plan for treating a region of interest. The radiation treatment plan includes a prescribed radiation dosage and patient anatomical data of the region of interested to be treated. The method also includes applying an inverse planning optimization model to determine an optimal thickness of an interior surface of the tandem applicator at a plurality of dwell positions within the region of interest. The method also includes generating a schedule of dwell times for the tandem applicator based on the generated position-dependent thickness profile. The method also includes transmitting design instructions to a 3D printer for fabrication of the tandem applicator.

### CROSS-REFERENCE TO RELATED APPLICATION

This application claims the benefit of U.S. Provisional Application Ser. No. 62/502,092, filed May 5, 2017, entitled RADIATION MODULATOR AND METHODS OF USE AND PRODUCTION THEREOF, which is hereby incorporated in its entirety herein.

### FIELD OF THE INVENTION

The present disclosure generally relates to a brachytherapy applicator, methods of producing the applicator, and methods of treatment using the applicator.

### BACKGROUND

High Dose Rate (HDR) brachytherapy, either paired with external beam radiation therapy (EBRT) or delivered alone, is a known treatment modality for cervical cancer at any stage. Like traditional EBRT, the dose delivered to the tumor in a brachytherapy treatment is limited by the presence of surrounding organs at risk (OARs). Existing brachytherapy tandem applicators typically include a single, central lumen and a source that is characterized by a highly isotropic dose profile. These characteristics limit the ability of these existing applicators to satisfy OAR dose constraints while simultaneously delivering the prescribed dose to the tumor. The limitations of these existing applicators are especially apparent in cases where the tumor is large, laterally extended, and/or anisotropically distributed.

Existing approaches for treating these extended and/or asymmetric tumors aim to create a more conformal dose profile by supplementing the intracavitary tandem with interstitial brachytherapy. Such approaches include using a modified tandem and ring applicators in which the ring also acts as a template for interstitial needles. Existing approaches further include intensity-modulated brachytherapy (IMBT) that enables anisotropic modulation of the source distribution, dynamic modulated brachytherapy in which the source is encapsulated by a cylindrical shield having a delivery window for radiation, rotating shield brachytherapy that makes uses of an applicator with an electronic brachytherapy source housed in a tandem applicator with an external rotating shield (e.g., rotating window), and direction modulated brachytherapy (DMBT) that makes use of an applicator with a source positioned on the periphery of the applicator as opposed to within a central lumen. Despite the improvements offered by these systems, many of these systems are complicated and may further increase the invasiveness of the treatment. Moreover, these approaches are ultimately still limited by the isotropic dose distribution of the source. Further, none of the existing approaches provide an applicator that is based on a patient's individual anatomy (e.g., the tumor size and position in relation to the position of the surrounding OARs), and is capable of delivering continuous and more conformal radiation dose profiles to extended and/or asymmetric tumors without harming the OARs. Rather, existing approaches utilize standard applicators that deliver doses of radiation that compromise coverage of the tumor (e.g., by covering only portions of the tumor) and/or cover the OARs.

Therefore, a need exists for a patient specific intensity-modulated HDR brachytherapy tandem applicator that yields conformal dose distributions, leads to improved target coverage compared to existing brachytherapy treatments, minimizes damage to the OARs, and improves radiation dose delivery time compared to the delivery time of existing brachytherapy treatments.

### BRIEF DESCRIPTION

In one aspect, a patient-specific intensity-modulated high dose rate (HDR) brachytherapy applicator for administering an HDR brachytherapy treatment to a patient is provided. The applicator includes a plurality of shielding segments distributed along a central longitudinal axis. Each shielding segment corresponds to one dwell position and includes a shielding wall. Each shielding wall includes a plurality of equiangular shielding sections of varying thickness distributed circumferentially about the central longitudinal axis. Each equilangular shielding section has a shielding thickness. Each shield thickness of each equiangular shielding section at each shielding segment is configured to transmit radiation from an HDR source positioned within each shielding segment into the patient at a predetermined dose rate distribution to administer the HDR brachytherapy treatment.

In another aspect, a computer-implemented method for designing a patient-specific intensity-modulated high dose rate (HDR) brachytherapy applicator for administering an HDR brachytherapy treatment to a patient is provided. The applicator includes a plurality of shielding segments distributed along a central longitudinal axis, and each shielding segment includes a plurality of equiangular shielding sections distributed circumferentially about the central longitudinal axis. The method is implemented using at least one processor in communication with at least one memory. The method includes receiving, by a computing device, a radiation treatment plan for administering the HDR brachytherapy treatment. The radiation treatment plan includes a prescribed radiation dosage to be delivered to a region of interest and patient anatomical data representative of the region of interest to be treated. The method also includes determining, by the computing device, an optimal shielding thickness profile and a plurality of optimal dwell times using an inverse planning optimization model constrained by the radiation treatment plan. Each optimal dwell time corresponds to one dwell position, and each dwell position corresponds to one shielding segment. The optimal thickness profile includes a plurality of shield thicknesses, and each shield thickness corresponds to one equiangular shielding section of one shielding segment. The method further includes generating a dwell position-dependent shielding thickness profile that includes the positions of the plurality of the shielding segments and each shield thickness of each equiangular shielding section at each shielding segment. The method additionally includes transmitting, by the computing device, design instructions to a three dimensional (3D) printer for fabrication of the applicator. The design instructions include at least the dwell position-dependent shielding thickness profile.

In an additional aspect, a computing device for designing a patient-specific intensity-modulated high dose rate (HDR) brachytherapy applicator for administering an HDR brachytherapy treatment to a patient is provided. The applicator includes a plurality of shielding segments distributed along a central longitudinal axis, and each shielding segment includes a plurality of equiangular shielding sections distributed circumferentially about the central longitudinal axis. The computing device includes at least one processor in communication with at least one memory device, and the at least one processor is programmed to receive a radiation treatment plan for administering the HDR brachytherapy treatment. The radiation treatment plan includes a prescribed radiation dosage to be delivered to a region of interest and patient anatomical data representative of the region of interest to be treated. The at least one processor is also programmed to determine an optimal shielding thickness profile and a plurality of optimal dwell times using an inverse planning optimization model constrained by the radiation treatment plan. Each optimal dwell time corresponds to one dwell position, each dwell position corresponds to one shielding segment, and the optimal thickness profile includes a plurality of shield thicknesses. Each shield thickness corresponds to one equiangular shielding section of one shielding segment. The at least one processor is further programmed to generate a dwell position-dependent shielding thickness profile that includes the positions of the plurality of the shielding segments and each shield thickness of each equiangular shielding section at each shielding segment. The at least one processor is additionally programmed to transmit design instructions to a three dimensional (3D) printer for fabrication of the applicator. The design instructions include at least the dwell position-dependent thickness profile.

In another additional aspect, a high-dose radiation (HDR) modulating system configured to improve target coverage of tumor volume during an HDR treatment is provided. The HDR modulating system includes a patient-specific intensity-modulated high dose rate (HDR) brachytherapy applicator that includes a plurality of shielding segments distributed along a central longitudinal axis. Each shielding segment includes a plurality of equiangular shielding sections distributed circumferentially about the central longitudinal axis. The plurality of shielding segments define a central lumen extending along the central longitudinal axis. Each shielding segment further defines a dwell position within the central lumen. The HDR modulating system also includes an HDR source that is movably insertable into the central lumen during an HDR treatment. The HDR source is configured to reside at each dwell position within each shielding segment for a corresponding dwell time. Each corresponding dwell time is based on a radiation therapy plan. Each equiangular shielding section at each shielding segment includes a shield thickness configured to transmit radiation from the HDR source residing at each dwell position at a predetermined dose rate distribution.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings illustrate various aspects of the disclosure.

FIG. 1A is an image showing an existing High Dose Rate (HDR) brachytherapy (BT) applicator being used to treat a patient with cervical cancer;

FIG. 1B is an illustration of an existing HDR BT applicator as shown in FIG. 1A being inserted into the cervical opening to treat cervical cancer;

FIG. 2 is an image showing existing HDR single dose distribution;

FIG. 3A is an anterior posterior (AP) image showing existing HDR sum dose distribution of all sources;

FIG. 3B is a lateral (LAT) image showing existing HDR sum dose distribution of all sources;

FIG. 4 is an image showing a screenshot of a GUI from an existing inverse planning software showing an isotropic radiation dosage distribution administered using an existing applicator design;

FIG. 5 is an illustration of an existing HDR technique directed at rotation/direction modulated brachytherapy (BT) for patient comfort;

FIG. 6A is an image showing an existing HDR applicator used to treat cervical cancer;

FIG. 6B is an illustration of an existing HDR technique termed direction modulated brachytherapy (DMBT) for improving patient comfort and target coverage;

FIG. 6C is a cross-sectional view of the tandem applicator of FIG. 6A having a single central lumen through which an HDR source delivers the prescribed radiation dose to the tumor;

FIG. 6D is a cross-sectional view of the DMBT applicator of FIG. 6B having six source positions around its periphery to improve delivery efficiency;

FIG. 6E is an intensity profile of the existing HDR BT applicator of FIG. 6C showing dose distribution;

FIG. 6F is an intensity profile of the DMBT applicator of FIG. 6D, showing dose distribution;

FIG. 7 is a cross-sectional view of a patient-specific HDR tandem applicator at one shielding segment, corresponding to one source dwell position, in accordance with one aspect of the disclosure;

FIG. 8A is a geometrical diagram illustrating relevant factors of a dose calculation in accordance with one aspect of the disclosure;

FIG. 8B is a graph showing cost functions for organs at risk (OARs) and the tumor based on the calculated dose Dᵢ;

FIG. 9 is an image showing a tungsten structure produced using a metal 3D printer in accordance with one aspect of the disclosure;

FIG. 10 is a block diagram schematically illustrating a computing system in accordance with one aspect of the disclosure;

FIG. 11 is a block diagram schematically illustrating a server system in accordance with one aspect of the disclosure;

FIG. 12 illustrates a diagram of components of a computing device configured for use with the computing system shown in FIG. 10;

FIG. 13 is a flow diagram illustrating an example method for designing a patient-specific HDR BT tandem applicator using the computing system shown in FIG. 10, in accordance with one aspect of the disclosure;

FIG. 14A is a side-view image of a the patient-specific intensity-modulated brachytherapy (IMBT) applicator in accordance with one aspect of the disclosure;

FIG. 14B is a side-view image of the patient-specific intensity-modulated brachytherapy (IMBT) applicator of FIG. 14A with the outer shielding layers removed to show the inner-most shielding layer;

FIG. 14C is perspective-view image showing a distal tip of the patient-specific intensity-modulated brachytherapy (IMBT) applicator of FIG. 14A, in accordance with one aspect of the disclosure;

FIG. 14D is a cross-sectional view of the patient-specific intensity-modulated brachytherapy (IMBT) applicator of FIG. 14A, showing the distribution of shielding at one shielding segment, corresponding to one dwell position, in accordance with one aspect of the disclosure;

FIG. 15A is a transparent perspective image of a patient-specific IMBT applicator as modeled in 3D printing software in accordance with one aspect of the disclosure, illustrating the distribution of shielding at several shielding segments/dwell positions;

FIG. 15B is a cross-sectional image looking along a longitudinal axis of the patient-specific IMBT applicator shown in FIG. 15A, in accordance with one aspect of the disclosure, illustrating the distribution of shielding at several shielding segments/dwell positions;

FIG. 16A is an image depicting a series of cross sections of a patient-specific IMBT applicator obtained at various dwell positions, in accordance with one aspect of the disclosure;

FIG. 16B is an image depicting a second series of cross sections of a patient-specific IMBT applicator obtained at various dwell positions, in accordance with one aspect of the disclosure;

FIG. 17A is an image showing a schematic illustration of an arrangement of the parameters of a dose rate calculation, in accordance with one aspect of the disclosure;

FIG. 17B is an image showing the schematic illustration of FIG. 17A with additional parameters of dose rate calculation, in accordance with one aspect of the disclosure;

FIG. 17C is an image showing the schematic illustration of FIG. 17A with additional parameters of dose rate calculation, in accordance with one aspect of the disclosure;

FIG. 18A is an image showing a dose rate map at a first dwell position within a patient-specific IMBT applicator, in accordance with one aspect of the disclosure;

FIG. 18B is an image depicting the modulation at a first dwell position within a patient-specific IMBT applicator corresponding to FIG. 18A, in accordance with one aspect of the disclosure;

FIG. 19A is a graph depicting a cost function for the tumor, in accordance with one aspect of the disclosure;

FIG. 19B is a graph depicting a cost function for an organ (e.g., the OARS), in accordance with one aspect of the disclosure;

FIG. 20 is a schematic illustration of a 2D phantom model used for initial experiments illustrating the clinical tumor volume (CTV) in relation to organ positions of the OARs;

FIG. 21 is a schematic illustration of a 2D patient data model used for experiments illustrating the CTV and organ positions of the OARs in a patient;

FIG. 22A is a graph of a transmission rate (down) for an existing tandem method estimated using the 2D phantom model of FIG. 20;

FIG. 22B is a graph of a transmission rate (up) for an existing tandem method estimated using the 2D phantom model of FIG. 20;

FIG. 22C is a graph of a dwell time for an existing tandem method estimated using the 2D phantom model of FIG. 20;

FIG. 23A is a graph of a transmission rate (down) for a patient-specific IMBT applicator method estimated using the 2D phantom model of FIG. 20;

FIG. 23B is a graph of a transmission rate (up) for a patient-specific IMBT applicator method estimated using the 2D phantom model of FIG. 20;

FIG. 23C is a graph of a dwell time for a patient-specific IMBT applicator method estimated using the 2D phantom model of FIG. 20;

FIG. 24A is an image showing an intensity profile for the existing tandem method showing the dose distributions estimated using the 2D phantom model;

FIG. 24B is an image showing an intensity profile for the patient-specific IMBT applicator method showing the dose distributions estimated using the 2D phantom model;

FIG. 25A shows the estimated dose distributions (e.g., dose coverages) overlaid on the 2D phantom model of FIG. 20 for the existing tandem method for prescribed doses of 550 cGy, 460 cGy, and 420 cGy;

FIG. 25B shows the estimated dose distributions (e.g., dose coverages) overlaid on the 2D phantom model of FIG. 20 for the patient-specific IMBT applicator method for prescribed doses of 550 cGy, 460 cGy, and 420 cGy;

FIG. 26A is a graph of a transmission rate (down) for an existing tandem method estimated using the 2D patient data of FIG. 21;

FIG. 26B is a graph of a transmission rate (up) for an existing tandem method estimated using the 2D patient data of FIG. 21;

FIG. 26C is a graph of a dwell time for an existing tandem method estimated using the 2D patient data of FIG. 21;

FIG. 27A is a graph of the transmission rate (down) for a patient-specific IMBT applicator method estimated using the 2D patient data of FIG. 21;

FIG. 27B is a graph of the transmission rate (up) for a patient-specific IMBT applicator method estimated using the 2D patient data of FIG. 21;

FIG. 27C is a graph of the dwell time for a patient-specific IMBT applicator method estimated using the 2D patient data of FIG. 21;

FIG. 28A is an image showing an intensity profile for an existing tandem method showing the dose distributions estimated using the 2D patient data of FIG. 21.

FIG. 28B an image showing an intensity profile for a patient-specific IMBT applicator method showing the dose distributions estimated using the 2D patient data of FIG. 21.

FIG. 29A shows the estimated dose distributions (e.g., dose coverages) overlaid on the 2D patient data of FIG. 21 for the existing tandem method for prescribed doses of 550 cGy, 460 cGy, and 420 cGy;

FIG. 29B shows the estimated dose distributions (e.g., dose coverages) overlaid on the 2D patient data of FIG. 21 for the patient-specific IMBT applicator method for prescribed doses of 550 cGy, 460 cGy, and 420 cGy;

FIG. 30A is an image of the axial dose distributions (e.g., dose coverages) at two slices for the 3D patient data using the existing tandem method at prescribed doses of 560 cGy, 460 cGy, and 420 cGy;

FIG. 30B is another image of the axial dose distributions (e.g., dose coverages) at two slices for the 3D patient data using the existing tandem method at prescribed doses of 560 cGy, 460 cGy, and 420 cGy;

FIG. 30C is an image of the dose distribution along the tandem axis for a single slice for the 3D patient data using the existing tandem method at prescribed doses of 560 cGy, 460 cGy, and 420 cGy;

FIG. 31A is an image of the axial dose distributions (e.g., dose coverages) at two slices for the 3D patient data corresponding to the image of FIG. 30A using the patient-specific IMBT applicator method at prescribed doses of 560 cGy, 460 cGy, and 420 cGy;

FIG. 31B is another image of the axial dose distributions (e.g., dose coverages) at two slices for the 3D patient data corresponding to the image of FIG. 30B using the patient-specific IMBT applicator method at prescribed doses of 560 cGy, 460 cGy, and 420 cGy;

FIG. 31C is an image of the dose distribution along the tandem axis for a single slice for the 3D patient data corresponding to the image of FIG. 30C using the patient-specific IMBT applicator method at prescribed doses of 560 cGy, 460 cGy, and 420 cGy;

FIG. 32 contains a series of images illustrating the DMBT treatment delivery method and conformality of the dose distribution in the DMBT delivery;

FIG. 33A is an image showing a representative image of a clinical rectal cancer case;

FIG. 33B is an image showing an example of a treatment for the clinical rectal cancer case of FIG. 33A as planned with a 7-field sliding-window IMRT plan using an existing Eclipse™ system;

FIG. 33C is an image showing an example of a treatment for the clinical rectal cancer case of FIG. 33A as planned with a DMBT system as shown in FIG. 32 in accordance with one aspect of the disclosure;

FIG. 34A is a perspective view of an existing paddle-based rotating shield brachytherapy (P-RSBT) applicator; and

FIG. 34B is a cross-sectional view of the applicator of FIG. 34A.

### DETAILED DESCRIPTION

The present disclosure relates to radiotherapy systems, devices, and methods for modulating the intensity of x-rays and/or gamma-rays emanating from a radiation source utilized to treat cancerous tumors. Such techniques can enable treatments that provide a non-invasive alternative to existing brachytherapy (CBT) treatments to treat cancerous tumors, such as cervical tumors. In various aspects, mathematical modeling is used for the optimization of a 3D printed patient-specific intensity-modulated brachytherapy (IMBT) applicator configured for use in high dose rate (HDR) treatments to successfully modulate radiation intensity to deliver focused radiation to a pathology site (e.g., gross tumor volume) while minimizing radiation exposure to surrounding organs at risk (OARs).

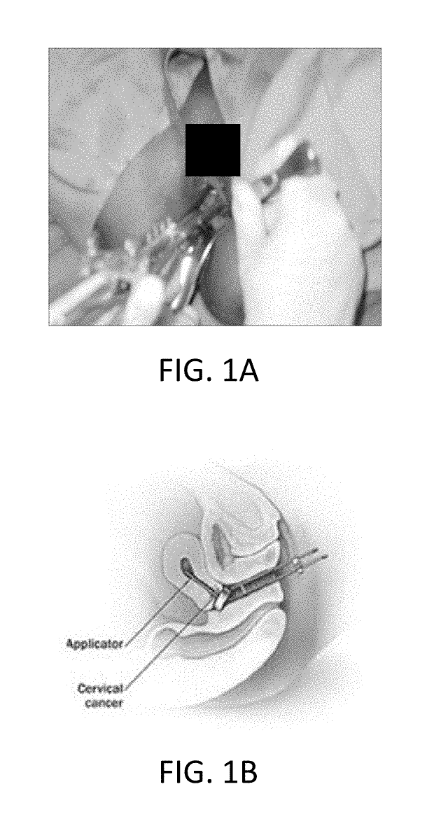

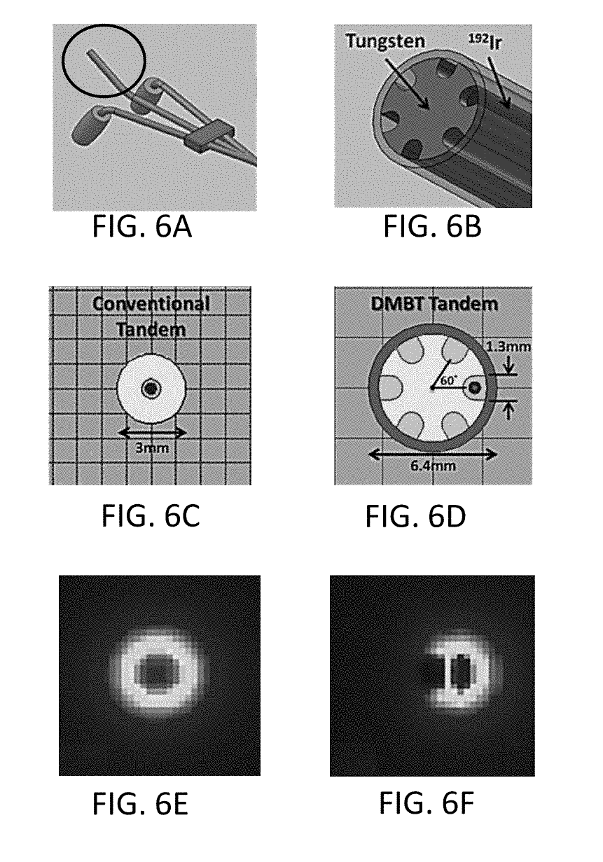

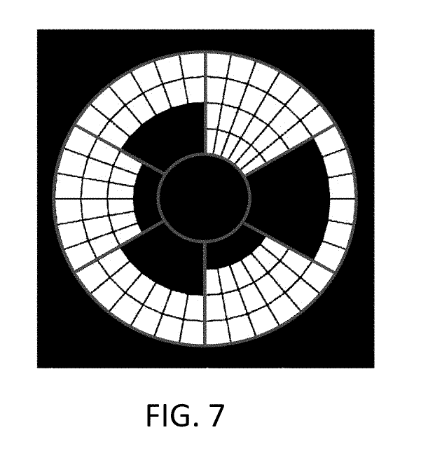

As described herein, the present disclosure is directed to patient-specific IMBT applicators for use in HDR treatments. In one aspect, the external shape of the applicator resembles an existing brachytherapy (BT) tandem applicator, as shown in FIGS. 1A, 1B, and 6A. More specifically, the external shape of the disclosed applicator in this aspect has a cylindrical profile similar to existing BT tandem applicators to ensure compatibility with existing applicators and associated treatment systems used in clinical practice. However, the internal shape of the present applicator is different from existing applicators in that the inner tandem wall of the applicator is divided into equiangular shielding sections of varying shielding thicknesses along a central longitudinal axis of the applicator at each radiation source dwell position (shown in FIGS. 7, 14C, 14D, and 15B). The thickness profiles of these sections, along with the dwell time at each dwell position, is optimized algorithmically using an alternating minimization scheme based on a specific patient's anatomical information and the radiation dosage prescribed by the patient's physician.

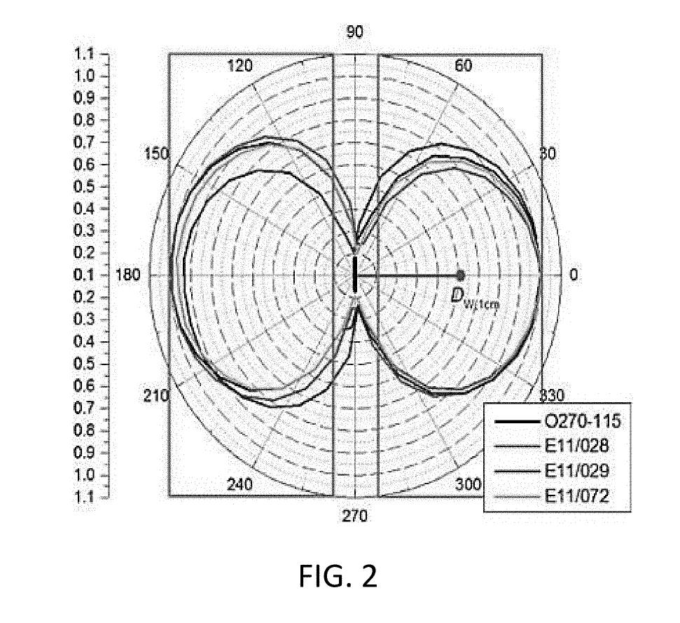

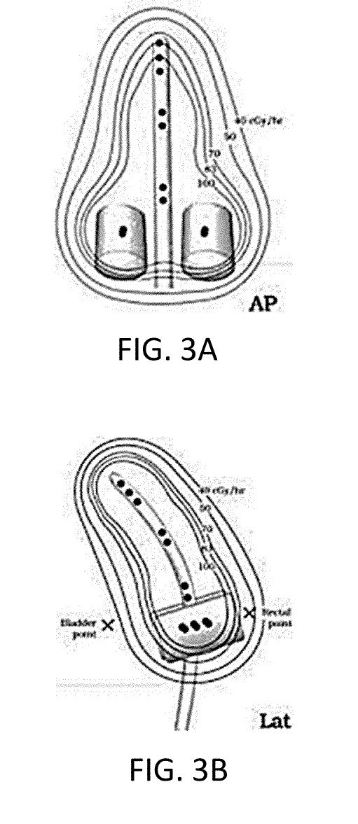

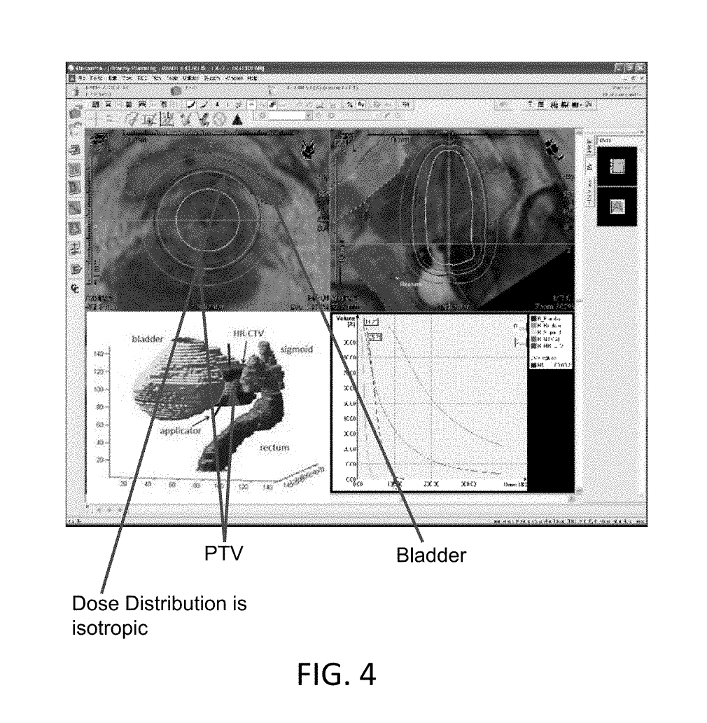

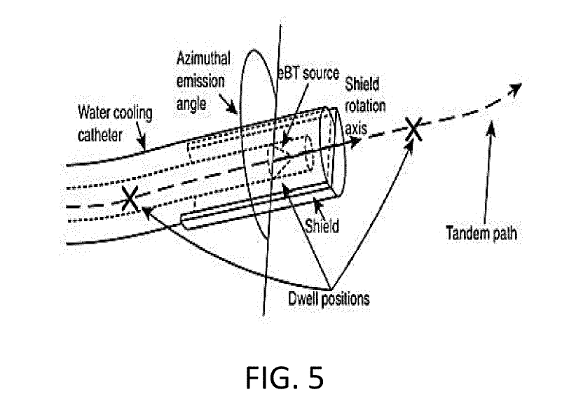

In contrast, existing BT tandem applicators consist of a single central lumen with a uniform cross-sectional profile, as shown in FIGS. 6C and 6E, through which an HDR source (e.g., a radiation source for HDR brachytherapy) is advanced and retracted to deliver dosages of radiation at a plurality of dwell positions. FIG. 2 illustrates a typical single dose distribution delivered by an existing BT tandem applicator. Similarly, FIGS. 3A and 3B provide images showing the dose profiles of all sources for an existing BT tandem applicator. Existing approaches for HDR BT treatment are accompanied by various limitations with respect to isotropic dose distribution during HDR BT treatment. As shown in FIG. 4, dose distributions may deliver radiation to the OARs (e.g., like the bladder) while providing minimum coverage to the tumor. Some existing BT approaches are designed to direct the emission from the HDR source to enable a greater degree of conformality. FIG. 5 illustrates a rotation modulated BT applicator directed to patient comfort. FIGS. 6B, 6D, and 6F illustrate a direction modulated brachytherapy (DMBT) applicator that includes six source positions around its periphery to improve delivery efficiency. Existing brachytherapy approaches known in the art are described in U.S. Patent Application Publication No. 2014/0249406, PCT International Publication No. 2014/021947, and U.S. Patent Application Publication No. No. 2016/0271379, all incorporated by reference herein in their entireties.

In various aspects, a patient's anatomical information includes information in regards to the patient's tumor. The patient's anatomical information can be provided by imaging modalities typically used for radiation therapy planning, such as, but not limited to, computerized tomography (CT) scans, ultrasonography scans, and magnetic resonance imaging (MRI) scans of the tumor and surrounding OARs. More specifically, the patient's anatomical information may include information defining the gross tumor volume (GTV), defined herein as the known tumor volume that can be seen, measured, and/or palpated. The patient's anatomical information may also include information defining the clinical target volume (CTV), defined herein as the volume of suspected tumor infiltration surrounding the GTV. The CTV can include the volume of suspected tumor extensions that may or may not be fully imaged and/or accurately defined.

In various aspects, the radiation dosage distributions may be generated by HDR sources such as isotopes including, but not limited to, ¹⁹²Ir, ¹³¹Cs, ¹²⁵I, ¹⁰³Pd, ¹⁹⁸Au, ¹⁸⁷W, ¹⁶⁹Yb, ¹⁴⁵Sm, ¹³⁷Cs, ¹⁰⁹Cd, ⁶⁵Zn, ¹⁵³Gd, ⁵⁷Co, ⁵⁶Co, and ⁵⁸Co.

In another aspect, radiation dosage distributions can be generated by an HDR source, such as an electronic brachytherapy (eBT) source contained within a novel modulator comprising of high-Z material (e.g., an atomic number “Z” that is greater than or equal to 22). Such isotopes can be referred to as, for example, non-electronic brachytherapy (BT) sources.

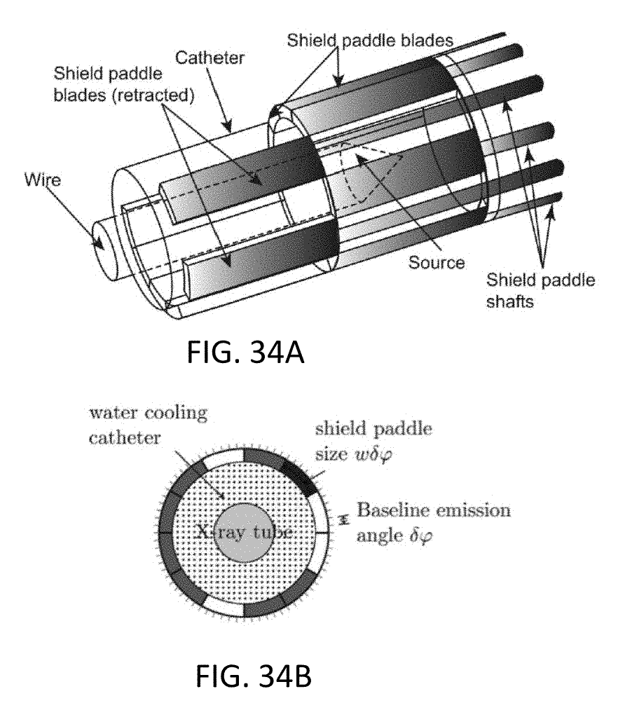

In one aspect, an individualized applicator, specifically the interior of the individualized applicator, is designed by optimizing wall thickness of the applicator and the dwell time based on a patient's anatomy and the prescribed treatment dosage. In this aspect, the interior of the individualized applicator is sub-divided into a plurality of equiangular shielding sections. Each section of the plurality of equiangular shielding sections may independently vary in shielding thickness at each dwell position of the applicator to enable anisotropic modulation by defining multiple emission windows at each dwell position, similar to paddle-based rotating shield brachytherapy (P-RSBT), shown illustrated in FIGS. 34A and 34B, without the extra complications associated with moving parts.

In one aspect, the thickness of the inner tandem applicator varies such that maximum radiation is delivered to tumor regions and minimal and/or zero radiation is delivered to nearby OARs. In another aspect, the disclosed applicator is optimized to modulate radiation intensity such that focused radiation is delivered to the GTV and the CTV. In various aspects, the number of equiangular shielding sections provided at each dwell position of the individualized applicator may depend upon a specific patient's anatomical information. By way of non-limiting example, the number of equiangular shielding sections can be increased to create a more spatially tailored dose distribution for a patient having a tumor that is laterally extended and/or anisotropically distributed. Increasing the number of equiangular shielding sections at each dwell position increases the computational cost of design optimization by the disclosed computer-implemented methods. However, the increased computation time is less than the computation time typically used for existing BT approaches. In another non-limiting example, the number of sections can be reduced for a patient depending on tumor position and/or volume with respect to OARs. Restricting the number of equiangular shielding sections at each dwell position reduces the computational cost of design optimization by the disclosed computer-implemented methods.

In another additional aspect, the number of equiangular shielding sections at each dwell position of the individualized applicator may be selected based on the resolution of the patient's anatomical information, as well as the estimated degree of movement of anatomical features of the patient during acquisition of the patient's anatomical information and/or during treatment. Without being limited to any particular theory, internal organs, tissues, and other anatomical landmarks are subject to a limited degree of movement. Consequently, the location of the tumor (i.e. GTV and CTV) and OARs at any given time in each patient may be subject to some degree of uncertainty. If a high number of equiangular shielding sections are included at each dwell position of the individualized applicator, the spatial resolution of the resulting radiotherapy dosage maps may be needlessly high given the lower degree of precision at which the patient's anatomical data is known.

In one aspect, the inner tandem wall of the applicator is divided into six equiangular shielding sections at each dwell position of the HDR source, as shown in FIG. 7. In various other aspects, the inner tandem wall of the applicator is divided into two equiangular shielding sections, three equiangular shielding sections, four equiangular shielding sections, five equiangular shielding sections, six equiangular shielding sections, seven equiangular shielding sections, eight equiangular shielding sections, nine equiangular shielding sections, ten equiangular shielding sections, or more equiangular shielding sections.

FIG. 7 illustrates an axial slice of the tandem applicator at one source dwell position. The tandem consists of six equiangular shielding sections at each dwell position. Each equiangular shielding section has an optimized thickness based on the specific patient's anatomical information. In one aspect, the exterior of the applicator includes an indicator such as a key, marker, and/or notch that is configured to orient the applicator such that prior to the treatment, the patient's attending physician and/or technician the appropriate position and/or orientation of the applicator to mount the applicator to the treatment device or system used to administer the radiotherapy using the applicator.

Optimization Model for Designing Patient-SpecificIntensity-Modulated HDR BT Applicator

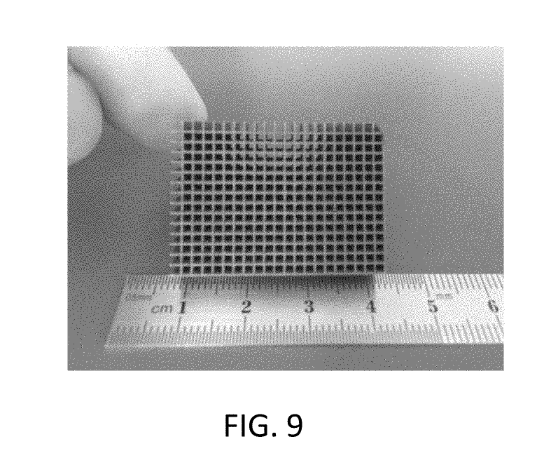

In various aspects, an optimization model is used to determine the thicknesses of the inner tandem wall (e.g., the shielding wall) of the applicator for each equiangular segment of each dwell position such that focused radiation (e.g., maximum radiation at the prescribed dosage) is delivered to the GTV and CTV, and minimum or no radiation is delivered to surrounding OARs. Applicator design parameters determined by the optimization model including, but not limited to, the inner wall thicknesses at each equiangular segment of each dwell position are input into 3D modeling software for 3D printing of the patient-specific intensity-modulated HDR BT tandem applicator. In various aspects, the 3D printed applicator is formed form a tungsten material (shown in FIG. 9). In various other aspects, 3D printed applicator may be formed from any suitable material without limitation, so long as the material provides sufficiently low transmission rates and is compatible for use with a 3D printing device. In the optimization model, the transmission rates of the shielding wall at each dwell position and the dwell time of the HDR source are variables that are optimized in order to achieve the best possible target coverage. In one aspect, the optimization model is a bi-convex optimization problem, and is solved using alternating minimization.

In one aspect, to compute the radiation dose rate, a 1D isotropic point source dose rate calculation formulation suggested by AAPM Report TG-43 is utilized. Specifically, the dose rate \dot{D}( [symbol] ) at a voxel at position [symbol] from a point source at position [symbol] is calculated as expressed in Equation 1:

$$\dot{D} ( \vec{r} ) = {S}_{K} \cdot Λ \cdot {( \frac{‖ \vec{{r}_{0}} ‖}{‖ \vec{r} ‖} )}^{2} \cdot {g}_{P} ( \vec{r} ) \cdot {φ}_{an} ( \vec{r} ) . \qquad ( Equation 1 )$$

where S_{K} denotes the air-kerma strength (units μG_{y}m²h⁻¹), Λ denotes the dose rate constant in water, g_{P}( [symbol] ) denotes the radial dose function of the point source, and ϕₐₙ( [symbol] ) denotes the 1D anisotropy function.

For the purpose of calculation, the volume of interest is discretized into voxels with resolution [rₓ mm×r_{y} mm×r_{z} mm] and index i. The dose rate in voxel i is subsequently induced by the j^{th} source, denoted by \dot{D}ᵢ ^{J}. Assuming there are Nₛ source positions located along the tandem separated by an equal distance dₛ and the tandem consists of Nₜ pieces of shielding, the total dose received by the i^{th} voxel is given by

$${D}_{i} = {\sum }_{j = 1}^{{N}_{s}} \dot{{D}_{i}^{j}} \cdot {t}_{j} \cdot {T}_{g ( i , j )}^{α ( i , j )} . \qquad ( Equation 2 )$$

In Equation 2, T_{g(i,j)}^{α(i,j)} is the transmission rate of a given piece of shield indexed by g(i,j) ranging from 1 to Nₜ, which is a function that determines which shield a ray originating at the j^{th} source will cross in travelling to the i^{th} voxel. In Equation 2, α(i, j) is a constant representing the ratio

$$\frac{‖ \vec{r} ‖}{‖ \vec{{r}_{⊥}} ‖} ,$$

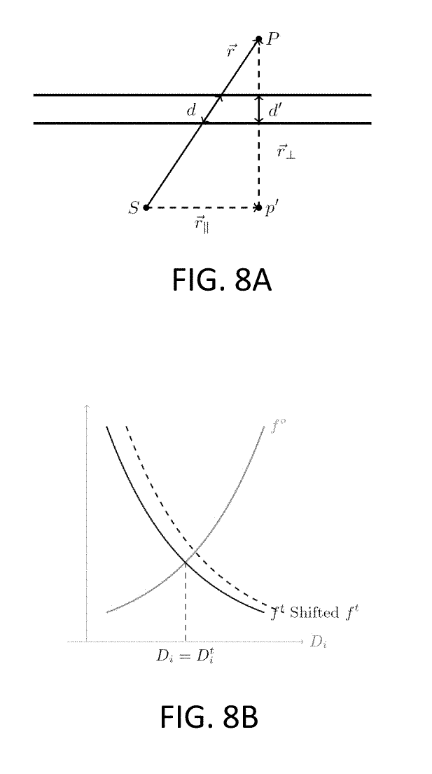

which reflects the effect of the thickness of the shield on the ray passing through it, as shown in FIG. 8A. The transmission factor of the shield is inversely proportional to its thickness, a factor that guides the design of the tandem before the 3D printing process. FIG. 8A is a geometrical representation of dose calculation where the vector [symbol] that travels through a length of shield d from source S to point of interest P is composed of parallel and perpendicular components, [symbol] and [symbol] respectively. In various aspects, the transmission factors are influenced by the desired dose distribution according to the specific anatomy of the patient and the prescription assigned by the physician.

In one aspect, to determine the dwell time for each dwell position and the transmission rate for each shielding portion, the following optimization model is utilized. In this optimization model, Nₚ dwell positions are assumed and t∈ [symbol] represents the vector of dwell times, and T∈ [symbol] represents the vector of transmission rates, where [symbol] ={t∈ [symbol] ^{N}^{ₚ}|tᵢ∈|0, +∞),i=1,2, . . . , Nₚ} and [symbol] ={T∈ [symbol] ^{N}^{ₛ}|Tᵢ∈[l, u],i=1,2, . . . , Nₛ}.

In this optimization model, l, u∈(0,1) are the lower and upper bounds of the transmission rate, which correspond to the thickness of each shielding portion. Considering the design limitations of the shield, l=0.38 and u=0.90 were set corresponding to thicknesses of 4.61 mm and 0.50 mm respectively. Subsequently, [symbol] ={1,2, . . . , Nₒ} represents the set of indexed OARs to be considered in the optimization method. [symbol] ₒ⊂ [symbol] represents the set of voxels making up the o-th OAR, while [symbol] ₜ⊂ [symbol] represents the set of voxels in the HR-CTV. The cost function for this optimization model is defined according to Equation 3 below:

$$F ( t , T ) = {\sum }_{o ∈ O} {F}_{o} ( t , T ) + {F}_{c} ( t , T ) = {\sum }_{o ∈ O} {\sum }_{i ∈ {V}_{o}} {f}_{i}^{o} ( t , T ) + {\sum }_{i ∈ {V}_{i}} {f}_{i}^{t} ( t , T ) . \qquad ( Equation 3 )$$

The first term in Equation 3 corresponds to the costs for OARs with the given configuration of dwell times t and the transmission rates T while the second term corresponds to the cost for the tumor. Additionally, the cost function at voxel level for the OARs is defined according to Equation 4 as follows:

$${f}_{i}^{o} ( t , T ) = exp ( \frac{{D}_{i} ( t , T ) - {D}_{i}^{t}}{C} + {S}_{o} ) , \qquad ( Equation 4 )$$

The cost function at voxel level for the tumor is defined according to Equation 5 as follows:

$${f}_{i}^{t} ( t , T ) = exp ( \frac{{D}_{i}^{t} - {D}_{i} ( t , T )}{C} + {S}_{t} ) , \qquad ( Equation 5 )$$

where Dᵢ(t,T) is the dose calculated using Equation 2, and Dₜ∈ [symbol] ^{N} represents the target dose for each voxel, which is defined as

$${D}_{i}^{t} = \{ \begin{matrix}{D}_{o}^{t} , & i ∈ {V}_{o} \\ {D}_{t}^{t} , & i ∈ {V}_{t} \\ 0 , & otherwise .\end{matrix} \qquad ( Equation 6 )$$

Here, Dₒ is the maximum dose that could be delivered to the o-th OAR while Dₜ is the minimum dose that should be delivered to the tumor. Additionally, in Equations 4 and 5, C∈ [symbol] is a constant that scales down the cost, while Sₒ and Sₜ are constants that control the relative importance for the o-th OAR or the tumor respectively. Unlike the general multi-objective optimization approach, in which a set of weighting parameters are utilized in presenting the total cost function as a convex combination of all the individual terms, horizontal shifting constants Sₒ and Sₜ are used to balance the relative importance of the individual cost functions. These cost functions are illustrated in FIG. 8B which depicts cost functions for OARs (green) and the tumor (blue) depending on the calculated dose Dᵢ. As shown in FIG. 8B, a larger Sₒ or Sₜ implies more weight on the corresponding term whether it is for an OAR or for the tumor. Without being limited to any particular theory, there are many appropriate choices for the cost function at the voxel level as long as the desired property is captured. In FIG. 8B, an exponential function form is chosen as an objective function for the purpose of demonstration.

The cost function presented in Equation 3 above may be expressed in a denser form to provide the optimization model given by Equation 7 below:

$$\begin{matrix}{min}_{t ∈ X , T ∈ y} F ( t , T ) \text{∷=} {\sum }_{i ∈ V} {f}_{i} ( t , T ) , & ( Equation 7 ) \\ {f}_{i} ( t , T ) = \{ \begin{matrix}{f}_{i}^{o} , & i ∈ {V}_{o} \\ {f}_{i}^{t} , & i ∈ {V}_{t} \\ 0 , & otherwise .\end{matrix} & ( Equation 8 )\end{matrix}$$

Equation 7 includes two blocks of variables and is convex for dwell time t when fixing transmission rates T, but not for the alternate case for transmission rates T when fixing dwell time t. To show this, it can be verified that fᵢ^{o}(t,T) is convex for one block of variables when fixing the other and fᵢ^{t}(t,T) is convex for t but concave for T. Specifically, D, is a linear function of t so it is both convex and concave, but it is convex for T as the Hessian is positive semidefinite. On the other hand, fᵢ^{o} is a convex non-decreasing function for t and T while fᵢ^{t} is convex and non-increasing. Since the summation of convex functions is convex, it follows that F is convex for t but the convexity with respect to T is not clear.

In one aspect, to solve Equation 7 an alternating minimization scheme is utilized to search for t and T in turns. This alternating minimization algorithm starts at an initial point (t₀, T₀)∈ [symbol] × [symbol] and solves the two subproblems with respect to t and T while fixing the other by gradient descent with back-tracking line search. Specifically, the partial gradients of fᵢ(t,T) with respect to the j^{th} dwell time is expressed as Equation 9 as follows:

$$\frac{δ}{δ {t}_{j}} {f}_{i} ( t , T ) = \{ \begin{matrix}\frac{{f}_{i} ( t , T ) {\dot{D}}_{l}^{J} {T}_{g ( i , j )}^{α ( i , j )}}{C} , & i ∈ {V}_{o} \\ - \frac{{f}_{i} ( t , T ) {\dot{D}}_{l}^{J} {T}_{g ( i , j )}^{α ( i , j )}}{C} , & i ∈ {V}_{t} \\ 0 , & otherwise ,\end{matrix} \qquad ( Equation 9 )$$

and partial gradients of fᵢ(t,T) for the k^{th} transmission rate T ₖ is expressed as Equation 10 below:

$$\frac{δ}{δ {T}_{j}} {f}_{i} ( t , T ) = \{ \begin{matrix}\frac{{f}_{i} ( t , T ) {\dot{D}}_{l}^{J} {t}_{j}}{C} , & i ∈ {V}_{o} , g ( i , j ) = k \\ - \frac{{f}_{i} ( t , T ) {\dot{D}}_{l}^{J} {t}_{j}}{C} , & i ∈ {V}_{t} , g ( i , j ) = k \\ 0 , & otherwise .\end{matrix} \qquad ( Equation 10 )$$

The gradient of F(t,T) with respect to t and T can be obtained by taking summations of Equations 9 and 10. Without being limited to any particular theory, the gradient descent method with constant step size bounded by

$$\frac{1}{L}$$

is unlikely to converge to a strict saddle point, where L is the Lipschitz constant for a function ƒ∈C². This conclusion does not necessarily hold for a gradient descent method with line search. In some aspects, a random perturbation to the iterate Tᵢ may be made when the step size is sufficiently small, or approximately less than the Lipschitz constant, and then the constant step size may be used to continue the optimization. The scheme of the algorithm in this aspect is summarized as shown below in Table 1.

TABLE 1
Algorithm 1 Alternating Minimization for Equation 7

| | |
|---|---|
| Input: | z⁰ = (t⁰, T⁰), (Sₒ)_{oϵO}, Sₜ, S_{T}, C, λₜ⁰, λ_{T}⁰, tolᵢₙ, tolₒᵤₜ, \hat{L}. |
| While | $\frac{‖ {z}^{n} - {z}^{n - 1} ‖}{‖ {z}^{n - 1} ‖} < {tol}_{out} do$ |
| 1. | Solve t subproblem: |
| | Set nₜ = 1, and do |
| | (a) λₜ = linesearch(F, tⁿ^{ₜ}, Tⁿ⁻¹, λₜ). |
| | $( b ) {t}^{{n}_{t}} = {t}^{{n}_{t} - 1} + {λ}_{t} \frac{\partial }{\partial t} F ( t , {T}^{n - 1} ) .$ |
| | $until \frac{‖ {t}^{{n}_{t}} - {t}^{{n}_{t} - 1} ‖}{‖ {t}^{{n}_{t} - 1} ‖} < {tol}_{i n} .$ |
| 2. | tⁿ = tⁿ^{ₜ}. |
| 3. | Solve T subproblem: |
| | Set n_{T} = 1, and do |
| | (a) if λₜ ≥ \hat{L}: |
| | λ_{T} = linesearch(F, tⁿ, Tⁿ^{_{T}}⁻¹, λ_{T}). |
| | else: |
| | Perturb Tⁿ^{_{T}} with a multivariate Gaussian noise n once. |
| | $( b ) {T}^{{n}_{T}} = {T}^{{n}_{T} - 1} + {λ}_{T} \frac{\partial }{\partial t} F ( {t}^{n} , T ) .$ |
| | $until \frac{‖ {T}^{{n}_{T}} - {T}^{{n}_{T} - 1} ‖}{‖ {T}^{{n}_{T} - 1} ‖} < {tol}_{i n} .$ |
| 4. | Tⁿ = Tⁿ^{_{T}}. |
| 5. | zⁿ = (tⁿ, Tⁿ), n = n + 1. |
| Output: | zⁿ = (tⁿ, Tⁿ). |

In one aspect, for user-specified set of constants Sₒ and Sₜ, Algorithm 1 will generate a plan with dwell time t and transmission rates T. However, the estimated dose volume histogram associated with this generated plan may not satisfy the clinical goal for tumor coverage and OAR dose. In another aspect, an automatic mechanism to tune the control constants Sₒ and Sₜ may be introduced to generate a satisfactory plan. In this other aspect, the algorithm assumes a relatively large Sₜ initially to ensure that the OAR sparing fails for at least some OARs, indicating that the current weighting of the cost function favors tumor coverage. Subsequently, the algorithm gradually increases Sₒ if the o-th OAR received excessive dose, and perform an additional iteration with the updated Sₒ. This procedure terminates when the OAR doses satisfy the prescribed criteria. This process of tuning the control constants Sₒ and Sₜ is summarized as Algorithm 2, shown below in Table 2. In various additional aspects, an initial guess of Sₜ may be used to tune Sₒ and Sₜ automatically in a manner similar to Algorithm 2.

TABLE 2
Algorithm 2 Automatic Search
Algorithm 2 Automatic search

```
Input: t⁰, T⁰, (Sₒ)_{o∈O}, Sₜ, δS, C, λₜ⁰, λ_{T}⁰, tolᵢₙ, tolₒᵤₜ.
While the calculated dose volume D does not satisfy the treatment target
do
    1. (tⁿ,Tⁿ) = Algorithm 1(tⁿ⁻¹,Tⁿ⁻¹,Sₒ,Sₜ,C,λₜ⁰,λ_{T}⁰).
    2. Calculate the dose volume based on t and T.
    3. for o = 1, ..., Nₒ, if D does not satisfy the condition for the o-th
    OAR
            Sₒ = Sₒ + δS.
Output: tⁿ,Tⁿ.
```

### Using the Calculated Parameters to Create a Patient-Specific Applicator

In various aspects, the optimal thickness of the shielding wall and the dwell time (e.g., the amount of time at which the HDR source delivers radiotherapy at each dwell position) are determined using the optimization model as described above. The optimal thickness and dwell time at each dwell position are determined according to the specific patient's anatomical information and the dosage prescribed by the patient's physician. As described above, the optimization model considers the transmission rates of the shielding wall of the tandem applicator, which depend on shielding thickness, and the dwell time of the HDR source as variables to be calculated in order to achieve the best possible target coverage. In various aspects, the best possible target coverage is where maximum radiation is delivered to the GTV and the CTV while minimum to no radiation is delivered to the OARs. By using alternating minimization to solve the optimization model described above, optimal thickness of the shielding wall and dwell time at each dwell position are calculated. In various aspects, these calculated parameters are subsequently input into 3D modeling software and used to 3D print an individualized applicator that, on the exterior, appears similar to existing applicators (e.g., cylindrical shape), but on the interior, differs in wall thickness based on the individual patient's anatomy and treatment needs. The determined thickness of the shielding wall at each dwell position is based on factors including, but not limited to, the thickness of the applicator inserted in the patient, the patient's anatomy, the patient's tolerance level, and whether or not certain degrees of thickness are suitable for continuous delivery of radiation. In various aspects, tungsten material, as shown in FIG. 9, is used to manufacture the applicator. By way of non-limited example, FIG. 9 illustrates a structure formed from a 3D printed tungsten material printed using a metal 3D printer (ProX DMP 320, 3D Systems, Belgium).

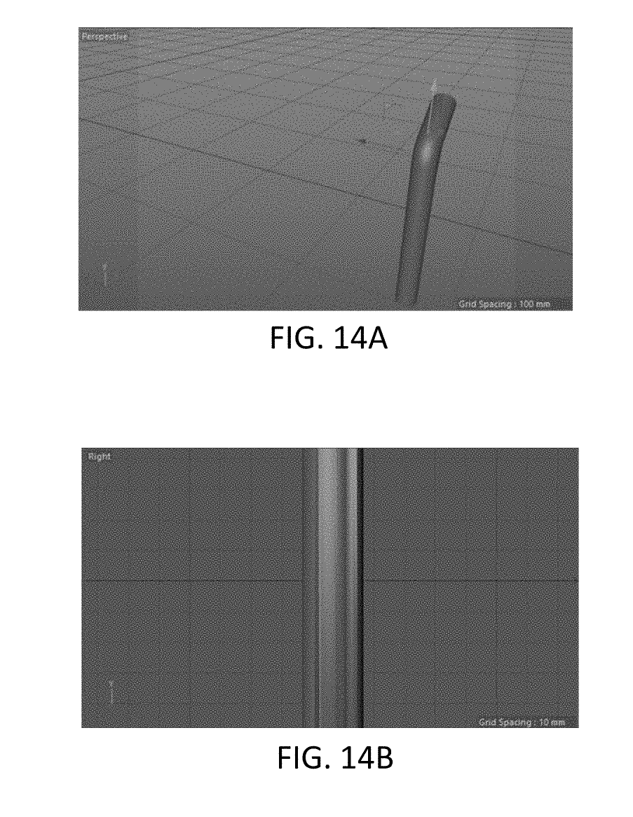

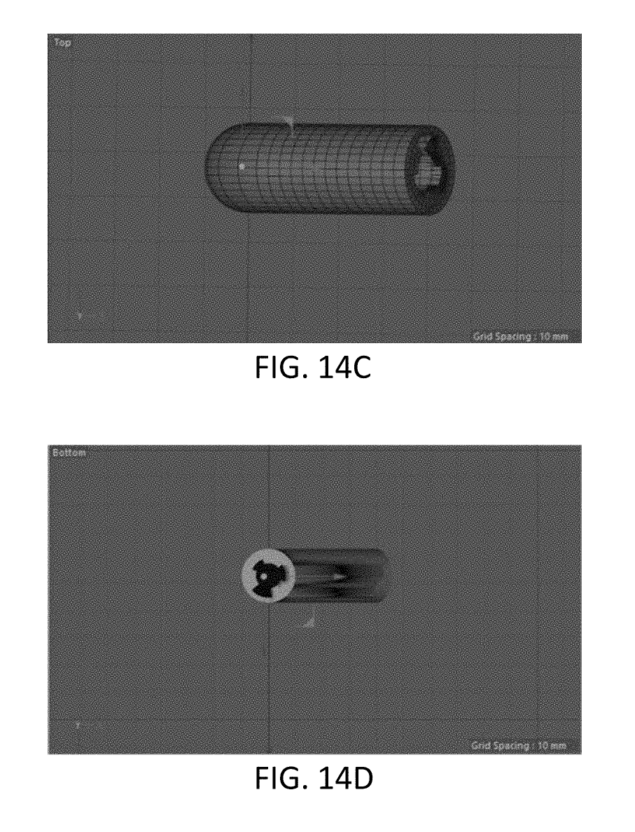

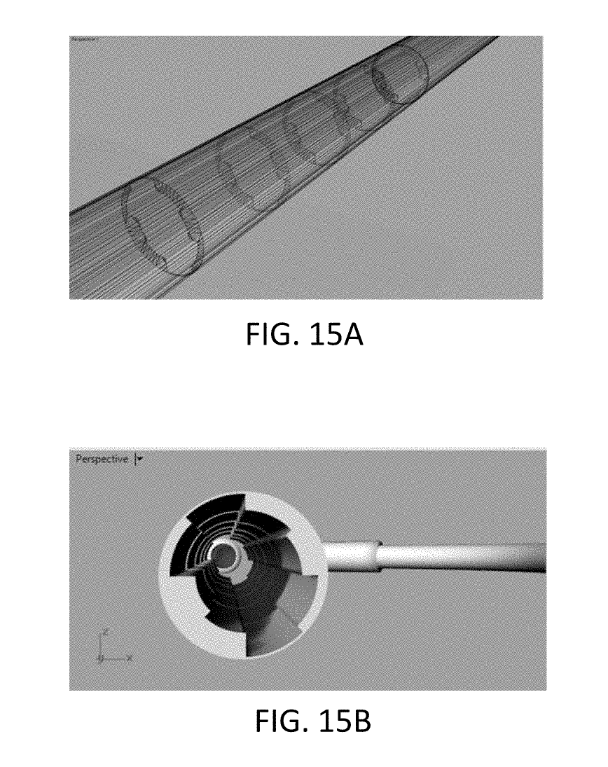

FIGS. 14A, 14B, 14C, and 14D illustrate various aspects of an individualized applicator design formed using the 3D printed tungsten material. FIGS. 14A, 14B, 14C, and 14D depict 3D modeling (Cinema 4D R17, Maxon) of an IMBT tandem applicator in one aspect that includes the design parameters calculated from the optimization model as described above. As shown in FIGS. 14C and 14D, the tandem applicator includes at least several different equiangular shielding sections with differing thicknesses distributed about the periphery of a circular inner lumen. In one aspect, the thickness of each equiangular shielding section varies from about 0.12 cm to about 0.48 cm. FIGS. 15A and 15B illustrate another patient-specific tandem applicator modeled using 3D design software for in fabrication of the applicator by a metal 3D printer in another aspect. In this other aspect, the patient-specific tandem applicator is designed to modulate the shielding thickness of the tandem applicator at each equiangular shielding section at each dwell position.

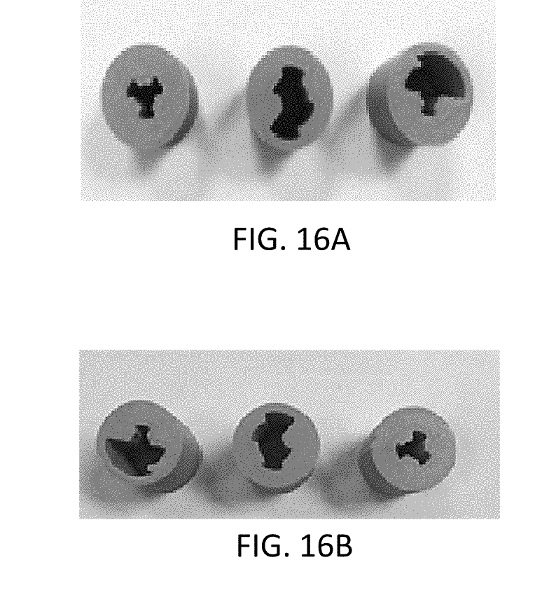

FIGS. 16A and 16B depict cross sections of an 3D printed IMBT tandem applicator at different dwell positions in an additional aspect. In this additional aspect each cross section shown in FIGS. 16A and 16B may be stacked together according to the corresponding dwell time of each cross-section to produce the 3D printed IMBT tandem applicator. Each cross section, corresponding to one dwell position, includes a shielding wall with a thickness divided into equiangular shielding sections. As seen in FIGS. 16A and 16B, the dwell positions have different thickness profiles through which the HDR source emanates radiation beams while residing at each dwell position for each predetermined dwell time. The cross sections shown in FIGS. 16A and 16B were 3D printed with polylactic acid (PLA) filament using a fused deposition modeling (FDM) 3D printer.

### Computing System

In some aspects, the above described methods and processes may be implemented using a computing system, including one or more computing devices. The methods and processes described herein may be implemented as computer applications, computer services, computer APIs, computer libraries, and/or any other computer program product without limitation.

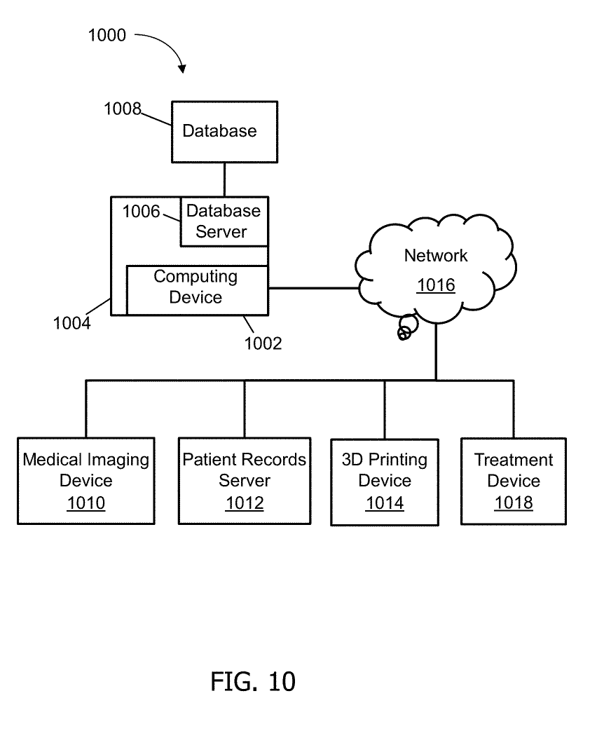

FIG. 10 is a simplified block diagram of a computer system 1000 for automatically designing a 3D-printed, patient-specific tandem applicator for intensity-modulated HDR brachytherapy in one aspect. The computer system 1000 may include a computing device 1002 configured to implement the patient-specific applicator design and fabrication methods disclosed herein. In one aspect, the computing device 1002 is part of a server system 1004, which also includes a database server 1006. The computing device 1002 is in communication with a database 1008 through the database server 1006. In one aspect, the database 1006 may include data such as, but not limited to, the inverse planning optimization model, applicator design parameters calculated from the model, recent imaging data (e.g., MRI, CT scans) of a patient to use in designing the applicator, radiation treatment plans providing information as to prescribed radiation dosages, patient medical history, historical image datasets of the tumor, and instruction sets to be transmitted to a 3D printing device 1014.

In some aspects, the computing device 1002 may be communicably coupled to at least one of a medical scanner 1010, a patient records server 1012, and a 3D printing device 1014 via a network 1016. In various aspects, the medical scanner 1010 can be any medical imaging system configured to obtain suitable images of a tumor (i.e. GTV and CTV) and organs (i.e. OARs) for analysis by the computing device 1002 including, but not limited to, an MRI scanner, a CT scanner, and any other suitable medical imaging device without limitation.

In various aspects, the computing device 1002 receives imaging data from medical scanner 1010 as well as a radiation treatment plan from the database 1008, and applies an optimization model to the imaging data and the radiation treatment plan to determine optimal thickness parameters for the applicator and dwell times at each dwell position, and transmits instructions to the 3D printing device 1014 for automatically creating a patient-specific applicator. The network 1016 may be any network that allows local area or wide area communication between the devices. For example, the network 1016 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem.

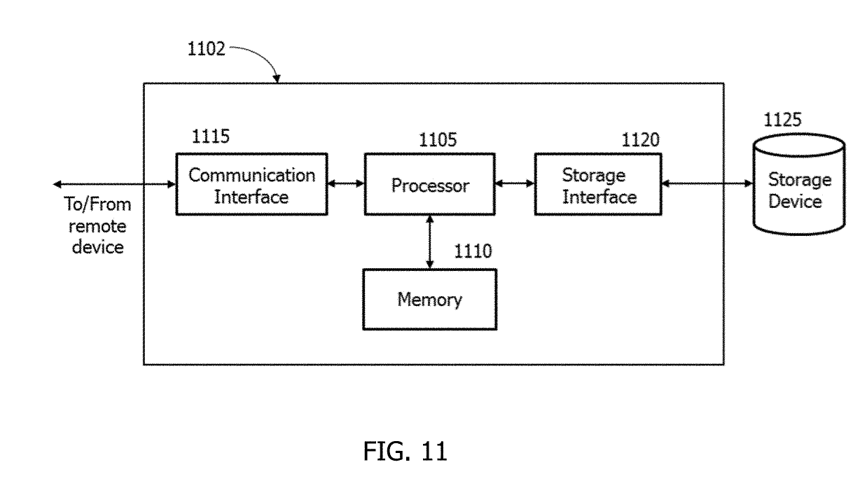

FIG. 11 illustrates one configuration of a server system 1102 in one aspect. The server system 1102 may include, but is not limited to, a database server 1006 and a computing device 1002 (both shown in FIG. 10). In some aspects, the server system 1102 is similar to the server system 1004 illustrated in FIG. 10. The server system 1102 may include a processor 1105 for executing instructions configured to enable the methods described herein. The instructions may be stored in a memory area 1110 in one aspect. The processor 1105 may include one or more processing units (e.g., in a multi-core configuration) in various aspects.

The processor 1105 may be operatively coupled to a communication interface 1115 such that the server system 1102 may be capable of communicating with a remote device such as a medical imaging device 1010, a patient records server 1012, a 3D printing device 1014, a treatment device 1018 (all shown in FIG. 10) and/or an additional server system. The communication interface 1115 may receive patient data from the medical imaging device 1010 and the patient records server 1012 via the network 1016 (see FIG. 10).

The processor 1105 may also be operatively coupled to a storage device 1125. The storage device 1125 may be any computer-operated hardware suitable for storing and/or retrieving data. In some aspects, the storage device 1125 may be integrated within the server system 1102. By way of non-limiting example, the server system 1102 may include storage device 1125 in the form of one or more hard disk drives. In other aspects, the storage device 1125 may be external to the server system 1102 and may be accessed by a plurality of server systems in addition to the server system 1102. By way of non-limiting example, the storage device 1125 may include multiple storage units including, but not limited to, hard disks or solid state disks in a redundant array of inexpensive disks (RAID) configuration. Other non-limiting examples of suitable storage devices 1125 include storage area networks (SAN) and/or network attached storage (NAS) systems.

In some aspects, the processor 1105 may be operatively coupled to the storage device 1125 via a storage interface 1120. The storage interface 1120 may be any component capable of providing the processor 1105 with access to the storage device 1125. Non-limiting examples of suitable storage interfaces 1120 include Advanced Technology Attachment (ATA) adapters, Serial ATA (SATA) adapters, Small Computer System Interface (SCSI) adapters, RAID controllers, SAN adapters, network adapters, and/or any suitable components providing the processor 1105 with access to the storage device 1125.

The memory 1110 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory suitable for storage of a computer program.

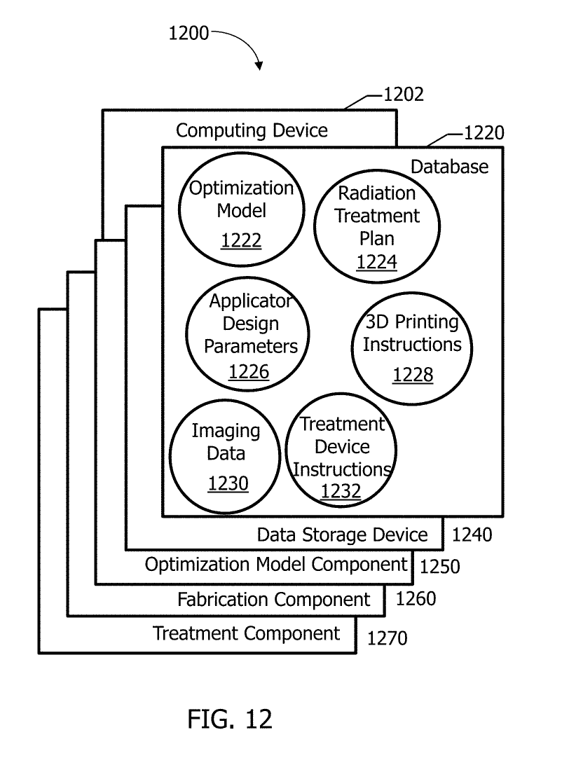

FIG. 12 depicts a component configuration 1200 of a computing device 1202, which includes a database 1220 along with other related computing components. In some aspects, the computing device 1202 is similar to the computing device 1002 shown in FIG. 10. In various aspects, a user may access components of computing device 1202 to implement the methods of designing and fabricating an patient-specific intensity-modulated HDR brachytherapy applicator and/or administer an HDR BT treatment using the patient-specific applicator as disclosed herein. In some aspects, the database 1220 is similar to the database 1008 shown in FIG. 10.

Referring again to FIG. 12, the database 1220 includes an optimization model 1222, a radiation treatment plan 1224, applicator design parameters 1226, 3D printing instructions 1228, imaging data 1230, and treatment device instructions 1232. The optimization model 1222 may include, but is not limited to, dose calculations for a region of interest, dose calculations for a dwell position, cost functions, and/or algorithms as described herein. Radiation treatment plan 1224 may include information to be used for radiation treatment planning including, but not limited to, radiation dose distribution maps for target tumor volumes, types of HDR sources to be used, anatomical information such as past imaging data of a patient's region of interest, patient file information, patient medical history, and prescription medication history. The imaging data 1230 may include current and/or most recent patient anatomical information in the form of one or more medical images. For example, imaging data 1230 may include the most recent MRI and/or CT scans that will be used to design the patient's tandem applicator using the optimization model as described above.

The applicator design parameters 1226 may include a plurality of parameters defining the geometry of the patient-specific applicator as calculated by the optimization model component 1260 using the optimization model 1222, including, but not limited to, a position of each dwell position along the applicator and thicknesses for each angular segment of each dwell position. The 3D printing instructions may include instructions generated by the fabrication component 1250 and based on the applicator design parameters 1226 to be used by a 3D printing device 1014 to fabricate the patient-specific applicator. The treatment device instructions 1232 may include instructions used by a treatment component 1270 to operate a treatment device 1018 to administer a treatment using the patient-specific applicator.

The computing device 1202 also includes at least several components configured to perform specific tasks associated with designing and producing a patient-specific applicator and to administer a treatment using the patient-specific applicator as disclosed herein. In one aspect, the computing device 1202 includes a data storage device 1240, an optimization model component 1250, a fabrication component 1260, and a treatment component 1270. The data storage device 1240 is configured to store data received or generated by computing device 1202, such as any of the data stored in database 1220 or any outputs of processes implemented by any component of computing device 1202.

The optimization model component 1250 is configured to receive a radiation treatment plan 1224 from the database 1220 for treating a region of interest. More specifically, the fabrication component 1260 is configured to receive a radiation treatment plan 1224 that includes, but is not limited to, a prescribed radiation dosage to be delivered to the region of interest and patient anatomical data of the region of interest to be treated for use in the design of the patient-specific applicator using the optimization model 1222. The optimization model component 1260 is configured to apply an inverse planning optimization model using the received prescribed radiation dosage and the received patient anatomical data (the radiation treatment plan 1224 and the imaging data 1230) to determine an optimal thickness of the interior shielding within the patient-specific applicator at a plurality of dwell positions within the region of interest. In addition, the optimization model component 1250 is configured to transmit the calculated plurality of dwell positions and associated shield thickness profiles for each dwell position to the fabrication component 1260. The optimization model component 1250 is further configured to transmit the plurality of dwell positions and associated dwell times to the treatment component 1270.

The fabrication component 1260 is configured to generate instructions used to operate the 3D printing device 1014 for fabrication of the patient-specific applicator. More specifically, the fabrication component 1260 is configured to receive design instructions from the optimization model component 1250 that include at least the plurality of dwell position and the associated shield thickness profiles.

The treatment component 1270 is configured to generate instructions that may be used to operate a treatment device 1018 to administer a treatment to a patient using the patient-specific applicator produced by the fabrication component 1260 using the 3D printing device 1014. In one aspect, the treatment component 1270 is configured to receive a plurality of dwell positions and associated schedule of dwell times for the patient-specific applicator from the optimization model component. The treatment fabrication component 1270 is further configured to modify each dwell time from the schedule of dwell times based on the age/condition of the radiation source using any method known in the art. In one aspect, the treatment component 1270 may control the operation of a treatment device 1018 including, but not limited to, a radiotherapy device, to administer a radiotherapy treatment to the patient using the patient-specific applicator.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

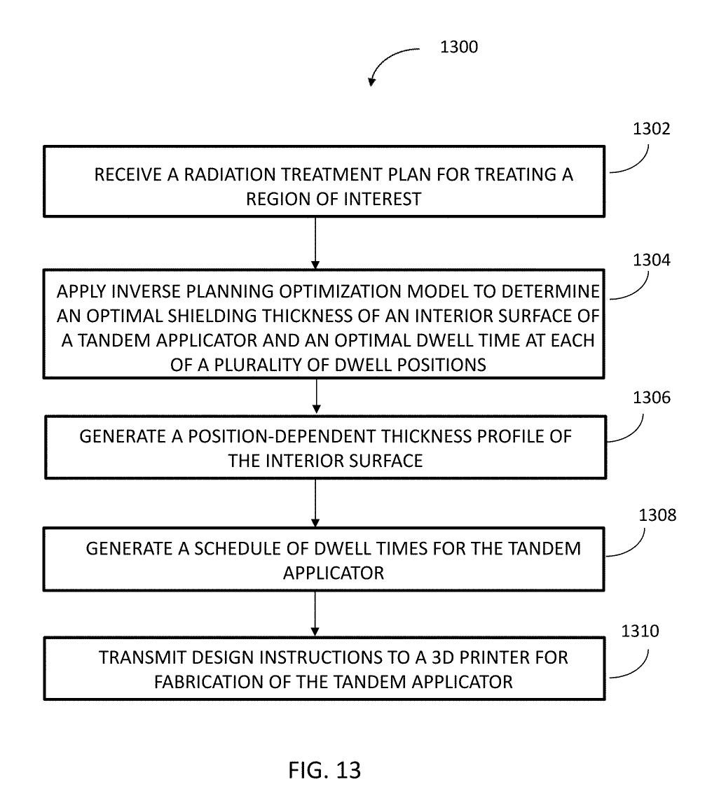

FIG. 13 illustrates a flow chart of a method 1300 for designing a patient-specific brachytherapy (BT) tandem applicator in one aspect. The method 1300 may be implemented by a computing device, such as computing device 1002 (shown in FIG. 10) and computing device 1202 (shown in FIG. 12). As illustrated in FIG. 13, the method 1300 includes receiving by a computing device, a radiation treatment plan for treating a region of interest at 1302. In one aspect, the radiation treatment plan includes, but is not limited to, a prescribed radiation dosage to be delivered to the region of interest and patient anatomical data of the region of interest to be treated. The method 1300 also includes applying, using the computing device, an inverse planning optimization model to determine an optimal thickness of an interior surface of the tandem applicator at a plurality of dwell positions within the region of interest at 1304. In various aspects, the inverse planning optimization model utilizes the received prescribed radiation dosage and the received patient anatomical data to optimize a shielding thickness and a dwell time at each dwell position. The method 1300 further includes generating, using the computing device, a position-dependent thickness profile of the interior surface of the tandem applicator based on the applied inverse planning optimization model at 1306. The method 1300 also includes generating, using the computer, a schedule of dwell times at 1308. In one aspect, the schedule of dwell times includes a plurality of dwell times, each dwell time associated with one dwell position within the tandem applicator as determined by the applied inverse planning optimization model. The method 1300 also includes transmitting, by the computing device, design instructions to a 3D printer at 1310 for fabrication of the tandem applicator. The design instructions include, but are not limited to, the dwell position-dependent thickness profiles generated at 1306. In one aspect, the method 1300 additionally includes transmitting, by the computing device, the schedule of dwell times generated at 1308 to a treatment device at 1312 for administration of a treatment using the tandem applicator fabricated using the design instructions transmitted at 1310.

In one embodiment, a computer program is provided, and the program is embodied on a computer-readable medium. In an example embodiment, the system is executed on a single computer system, without requiring a connection to a server computer. In a further example embodiment, the system is being run in a Windows® environment (Windows is a registered trademark of Microsoft Corporation, Redmond, Wash.). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, Calif.). In yet a further embodiment, the system is run on a Mac OS® environment (Mac OS is a registered trademark of Apple Inc. located in Cupertino, Calif.). In still yet a further embodiment, the system is run on Android® OS (Android is a registered trademark of Google, Inc. of Mountain View, Calif.). In another embodiment, the system is run on Linux® OS (Linux is a registered trademark of Linus Torvalds of Boston, Mass.). The application is flexible and designed to run in various different environments without compromising any major functionality. The following detailed description illustrates embodiments of the disclosure by way of example and not by way of limitation. It is contemplated that the disclosure has general application to providing an on-demand ecosystem in industrial, commercial, and residential applications.

The methods and systems described herein can be used to treat any disease, disorder, or condition that can be treated with traditional brachytherapy. For example, diseases, disorders, and/or conditions can include pathology, tumor, and cancer such as, but not limited to, prostate cancer, breast cancer, lung cancer, esophageal cancer, gynecologic cancer, anal/rectal tumor, sarcoma; and head or neck cancer. The tumor can be cancerous or non-cancerous lesions.

The present devices and methods enable treatment of lateral tumor extensions by delivering targeted radiation dosages to these areas. Lateral tumor extensions are difficult to treat with existing intracavitary brachytherapy (BT) due to radiation dose limitations imposed by the presence of nearby healthy tissues and organs, such as the bladder, rectum, and/or sigmoid in the case of cervical cancer treatment. In another aspect, the present devices and methods can enable increased dosage conformity for non-symmetric tumors by utilizing a device that can shield radiation emanated from an electronic brachytherapy (eBT) source or non-electronic brachytherapy (BT) source. In one example, the device includes a radiation modulator that includes a material having a position-dependent thickness that is based at least on (i) a radiation therapy plan specific to a patient and (ii) a geometry of a patient region to be treated (e.g., tumor region). In an additional or alternative aspect, the device includes an HDR source that is movably inserted into an enclosure coupled to the radiation modulator. In various aspects, the methods as described herein can include, the HDR source residing at a plurality of locations within the radiation modulator during a respective plurality of dwell times based on a patient's radiation therapy plan.

### Therapeutic Methods

Also provided is a process of treating a pathology, cancer, or tumor in a subject in need administration of a therapeutically effective amount of radiation, so as to destroy pathologic cells and shrink tumors.

Methods described herein are generally performed on a subject in need thereof. A subject in need of the therapeutic methods described herein can be a subject having, diagnosed with, suspected of having, or at risk for developing pathologic cells, tumors, or cancer. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of radiation is, for example, that amount that would cause the desired therapeutic effect in a subject while minimizing undesired side effects. In various embodiments, an effective amount of radiation described herein can substantially inhibit tumor or pathologic cell growth, slow the progress of tumor or pathologic cell growth, or limit the development of tumor or pathologic cell growth.

When used in the treatments described herein, a therapeutically effective amount of radiation can be any amount as prescribed by a radiologist.

Again, each of the states, diseases, disorders, and conditions, described herein, as well as others, can benefit from the treatment methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of radiation can occur as a single event or over a time course of treatment. For example, radiation can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more. As another example, a radiation delivery device can be implanted.

Treatment in accord with the methods described herein can be performed prior to, concurrent with, or after existing treatment modalities for tumor or pathologic cell (e.g., cancer) growth.

### Administration

Radiation treatment, as described herein, can be administered according to methods described herein and in a variety of means known to the art (see e.g., U.S. Patent Application Publication No. 2014/0249406; U.S. Patent Application Publication No. 2015/0367144; and U.S. Patent Application Publication No. 2016/0271379, incorporated by reference in their entireties herein).

As discussed above, radiation therapy can be administered in a dose or a plurality of doses or the radiation can be delivered via an implant.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to software, 3D printing materials, or a 3D printer. Such packaging of the components separately can, if desired, be presented in a pack or dispenser device which may contain one or more unit dosage forms containing the composition. The pack may, for example, comprise metal or plastic foil such as a blister pack.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

As employed in this specification and annexed drawings, the terms “unit,” “component,” “interface,” “system,” “platform,” “stage,” and the like are intended to include a computer-related entity or an entity related to an operational apparatus with one or more specific functionalities, wherein the computer-related entity or the entity related to the operational apparatus can be either hardware, a combination of hardware and software, software, or software in execution. One or more of such entities are also referred to as “functional elements.” As an example, a unit may be, but is not limited to being, a process running on a processor, a processor, an object, an executable computer program, a thread of execution, a program, a memory (e.g., a hard disc drive), and/or a computer. As another example, a unit can be an apparatus with specific functionality provided by mechanical parts operated by electric or electronic circuitry which is operated by a software or a firmware application executed by a processor, wherein the processor can be internal or external to the apparatus and executes at least a part of the software or firmware application. In addition or in the alternative, a unit can provide specific functionality based on physical structure or specific arrangement of hardware elements. As yet another example, a unit can be an apparatus that provides specific functionality through electronic functional elements without mechanical parts, the electronic functional elements can include a processor therein to execute software or firmware that provides at least in part the functionality of the electronic functional elements. An illustration of such apparatus can be control circuitry, such as a programmable logic controller. The foregoing example and related illustrations are but a few examples and are not intended to be limiting. Moreover, while such illustrations are presented for a unit, the foregoing examples also apply to a component, a system, a platform, and the like. It is noted that in certain embodiments, or in connection with certain aspects or features thereof, the terms “unit,” “component,” “system,” “interface,” “platform” can be utilized interchangeably.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches that may function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Validation of a Method for Fabricating a Patient-specific IMBT Tandem HDR Applicator for Cervical Cancer Using a 3D Printer

To validate the methods described above, the following experiments were conducted. A tandem applicator was designed such that the external shape of the patient-specific tandem applicator resembles the existing tandem applicator's external shape (e.g., a cylindrical shape). The wall thickness inside the patient-specific tandem applicator and the dwell time at each dwell position p were simultaneously optimized to provide varying degrees of thickness around each circumference at each dwell position based on the specific patient's anatomy and the prescribed radiation dosage.

Tungsten material was used to 3D print the patient-specific tandem applicator. To generate a model for 3D printing, an optimization model as described above was solved to generate the design parameters of the patient-specific tandem applicator. More specifically, the optimization model was solved to generate the transmission rate relating to tungsten thickness for a given schedule of dwell times.

The following cost function was used to calculate the optimization model parameters:

$${f}_{total} ( X ) = {f}_{organ} ( X ) + {f}_{tumor} ( X ) = {\sum }_{r = 1}^{3} {\sum }_{i = 1}^{N} {e}^{\frac{{X}_{r} ( i )}{c} + {S}_{r}} + {\sum }_{i}^{N} {e}^{- \frac{{X}_{4} ( i )}{c} + {S}_{4}} \qquad ( Equation 11 )$$

where Xᵣ(i)=Σₚ^{K}tₚDₚ(i)T_{g(i,p)}^{α(i,g(i,p))}−D_{o,r}(i), D=dose, p=dwell position, t is the dwell time, and T=transmission factor at each specific angular section.

After optimizing the cost function, the model parameters were inputted into 3D modeling software (Cinema 4D R17, Maxon) to generate a stereolithographic (STL) file for use by the 3D printing device.

At each dwell position of the HDR source, the surrounding tandem wall was divided into sections or segments with varying thicknesses. The thickness of each section varied from 0.12 cm to 0.48 cm inwards, such that the exterior surface of the tandem applicator resembled the traditional applicator (diameter=1.2 cm). The inner wall thickness profiles were generated using the ‘inner extrude’ function in the 3D modeling software for all pre-defined dwell positions. The resulting 3D model was then exported to the 3D printing software (Simplify3D) and converted from 3D volumetric data into an STL file. A 3D metal printer (ProX DMP 320) was utilized to fabricate the tandem applicator using tungsten, as shown in FIG. 9. Tandem samples, as shown in FIGS. 16A and 16B, were printed by a fused deposition modeling (FDM) 3D printer with polylactic acid (PLA) filament to verify the accuracy and uncertainty of the 3D printing process.

3D printing the patient-specific tandem applicator using design parameters calculated from the optimization model described above was achieved without any major discrepancies between the digital and physical models. A comparison between the model parameters and measurements from the 3D printed model of the applicator indicated an accuracy within about 0.1 mm.

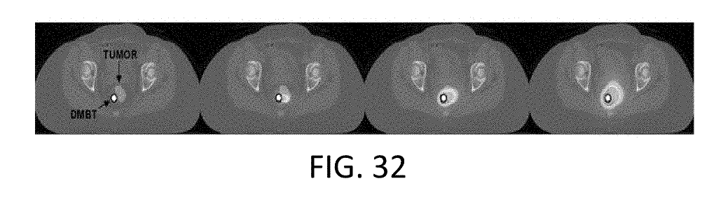

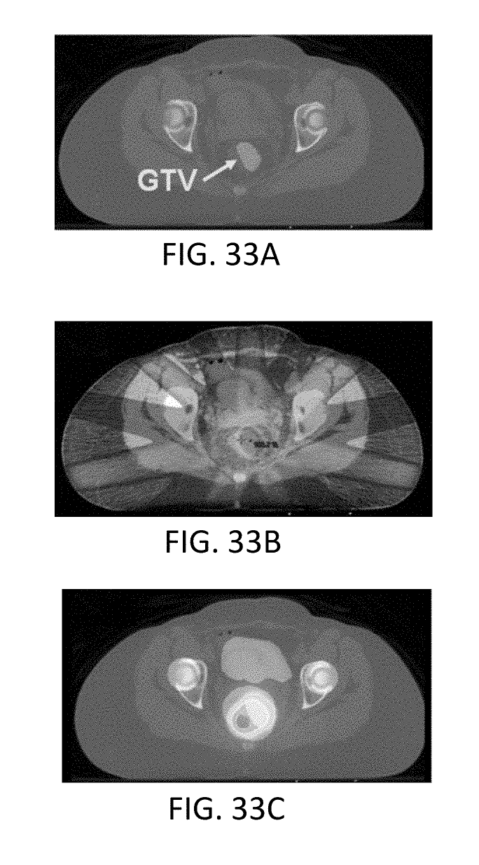

The disclosed method of utilizing an inverse planning optimization model to design a patient-specific tandem applicator was demonstrated to be a feasible alternative to existing tandem applicators, especially when surrounding OARS significantly constrain the tumor-dose coverage during HDR. Further, the disclosed method may be adapted to other HDR sites such as rectal (shown in FIGS. 32, 33A, 33B, and 33C), prostate, and breast. FIG. 32 illustrates, from left to right, a collimated radiation beam from an existing applicator design that included a shield, such as the applicator shown in FIG. 5, housing a Ir-192 source during treatment. The example applicator rotated on its axis at planned dwell times to expose the tumor volume to a prescribed dose. FIGS. 33A, 33B, and 33C show a series of images depicting an example clinical rectal cancer case. More specifically, FIG. 33B shows an example rectal cancer case planned with a 7-field sliding-window IMRT plan using the Eclipse™ system and FIG. 33C shows the example clinical rectal cancer case planned with the system shown in FIG. 5.

### Example 2

### Validation of a Method for Designing a Patient-specific IMBT Tandem HDR Applicator Using an Inverse Planning Optimization Model

To validate the method described above, the following experiments were conducted.

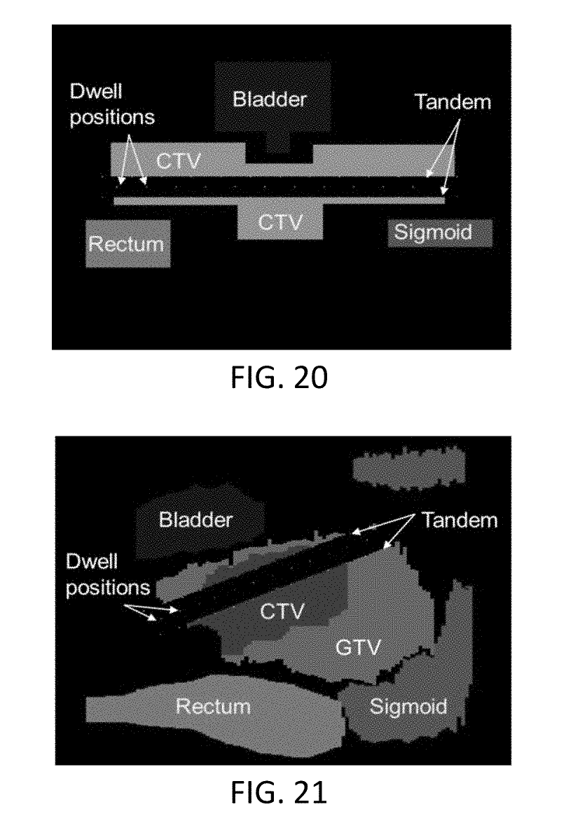

Numerical experiments were performed on a 2D phantom, shown in FIG. 20. The 2D phantom consisted of a clinical target volume (CTV) positioned on either side of the tandem applicator and three OARS: a bladder, rectum, and sigmoid. As illustrated in FIG. 20, the CTV surrounded the tandem applicator on either side. The three OARs were taken into consideration for optimization.

Numerical experiments were also performed using 2D patient data from a clinically-treated cervical cancer patient for further validation. FIG. 21 illustrates the 2D patient data. In this case, the tandem applicator was surrounded by the CTV, GTV, and the same three OARs were considered for optimization as were considered in the 2D phantom shown in FIG. 20.

Numerical experiments were also performed using 3D patient data from a clinically treated cervical cancer patient for further validation.

### Dose Rate Calculation (Based on AAPM TG-43 Report)

The following dose rate calculation formulation, based on the formulation described in the AAPM TG-43 Report, the contents of which are incorporated by reference herein in its entirety, was used to calculate the dose rate for the region of interest, as expressed in Equation 12:

$$\dot{D} i ( r , θ ) = {S}_{K} \cdot Λ \cdot ( \frac{G ( r , θ )}{G ( {r}_{0} , {θ}_{0}} ) \cdot g ( r ) \cdot F ( r , θ ) \qquad ( Equation 12 )$$

The dose at one dwell position was subsequently calculated using the following:

$$D ( r , θ ) = {\sum }_{i = 1}^{N} {\dot{D}}_{l} ( r , θ ) x {t}_{i} x {T}_{i} ( r , θ ) \qquad ( Equation 13 )$$

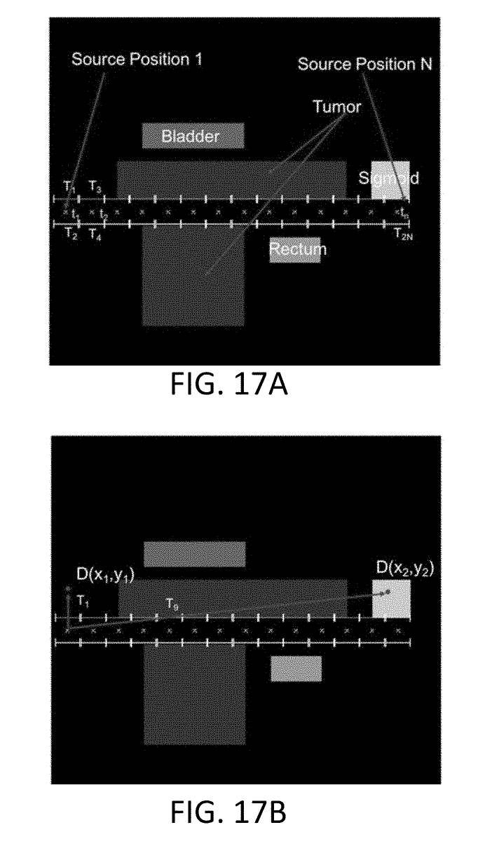

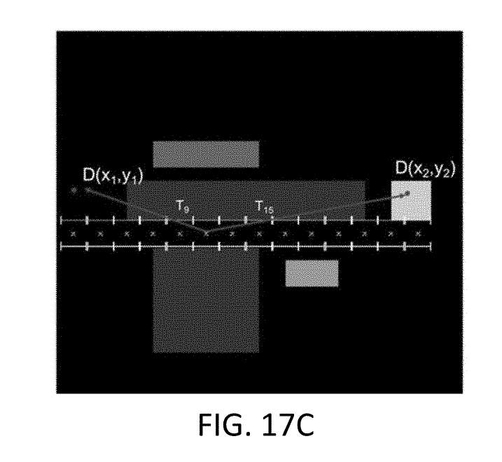

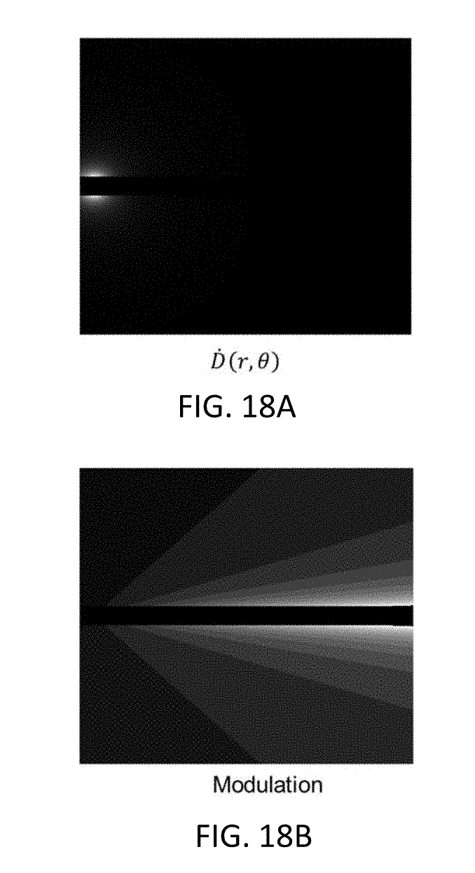

In Equation 13, tᵢ is the HDR source dwell time for each dwell position in seconds, Tᵢ is the transmission factor (e.g., the transmission rate) of the HDR source, and 0.25 (Maximum Thickness)≤Tᵢ≤0.8 (Minimum thickness). The transmission factor Tᵢ of the HDR source is related to the thickness of the shield wall of the tandem applicator. FIGS. 17A, 17B, and 17C illustrate the parameters used for the dose rate calculation of Equation 13. FIG. 18A depicts an estimated dose rate map at the first dwell position. FIG. 18B depicts an estimated radiation modulation at the first dwell position.

IMRT optimization was performed using Equation 14 below:

fₜₒₜₐₗ(D_{c})=fₜᵤₘₒᵣ(D_{c})+f_{rectum}(D_{c})+f_{bladder}(D_{c})+f_{sigmoid}(D_{c}) (Equation 14)

In Equation 14, D_{c} is the calculated dose of a 2D or 3D matrix depending on the dwell time tᵢ and the transmission rate Tᵢ.

The exponential cost function of Equation 15 was chosen for this optimization:

$$f ( {D}_{c} ) = {\sum }_{i = 1}^{N} {e}^{\frac{{D}_{c} ( i ) - {D}_{o} ( i )}{c} + S} \qquad ( Equation 15 )$$

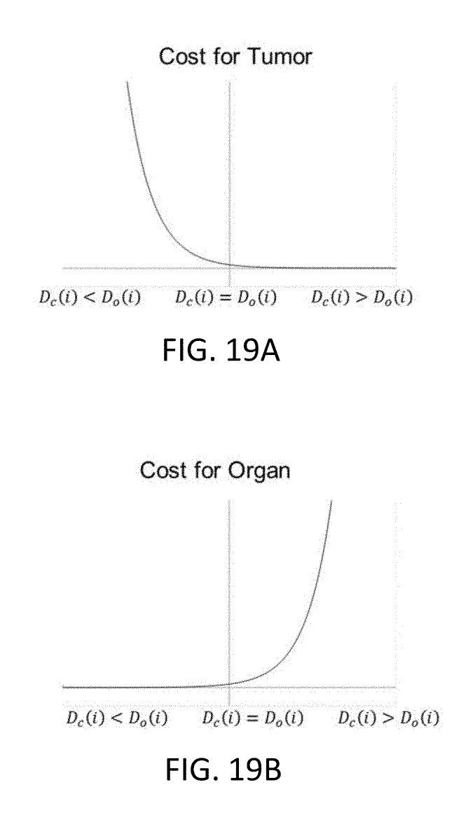

FIGS. 19A and 19B illustrate a series of graphs depicting the IMRT optimization model of Equation 14. More specifically, FIG. 19A illustrates the cost function for the tumor, which depended on the calculated doseD_{c}(i). FIG. 19B illustrates the cost function for the organs (e.g., OARs). The cost functions illustrated in FIGS. 19A and 19B were similar to the cost functions shown in FIG. 8B.

An objective function was utilized as expressed in Equations 16 and

$$\begin{matrix}{f}_{total} ( X ) = \underbrace{{\sum }_{r = 1}^{3} {\sum }_{r = 1}^{N} {e}^{\frac{{x}_{r} ( i )}{c} + {S}_{r}}}_{Organ} + \underbrace{{\sum }_{i}^{N} {e}^{- \frac{{x}_{4} ( i )}{c} + {S}_{4}} 17}_{Tumor} \text{:} & ( Equation 16 ) \\ {X}_{r} ( i ) = {\sum }_{p}^{K} {t}_{p} {D}_{p} ( i ) {T}_{g ( i , p )}^{∝ ( i , g ( i , p ) )} - {D}_{o , r} ( i ) & ( Equation 17 )\end{matrix}$$

For the objective functions, tₚ was dwell time at location p, Tⱼ was the transmission rate at index j, and both tₚ and Tⱼ were varied during optimization. Dₚ is the dose rate matrix at dwell location p. Additionally, c and sᵣ are constants that control the shape of the cost function. Further, g(i,p) is the index of the transmission rate, which depends on the dwell location p and the pixel location, and α(i,g(i,p)) is a constant that depends on the pixel location and the index of transmission rate.

Both the dwell time and the thickness of the equiangular shielding sections of the tandem applicator were optimized using an alternating minimization scheme to search for tₚ and Tⱼ. More specifically, alternative minimization with gradient descent and back-tracking line search was utilized using the method as follows:

STEP 1. Update the dwell time:

β=Linesearch(f,t^{k-1}, T^{k-1})

t^{k}=t^{k-1}+β∇f(t,T^{k-1}) (Equation 18)

STEP 2. Update the transmission rate:

γ=Linesearch(f,t^{k}, T^{k-1})

T^{k}=T^{k-1}+γ∇f(t^{k},T) (Equation 19)

STEP 3. Check convergence, if there is no convergence, go to STEP 1; otherwise go to STEP 4.

STEP 4. Check the constraints as expressed in Equation 20 below. If the constraints are not satisfied, modify the model by changing sᵣ and proceed to STEP 1. If the constraints are satisfied, terminate.

$$\frac{\partial {f}_{organ}}{\partial {t}_{p}} = {\sum }_{p}^{K} {\sum }_{i}^{N} \frac{{D}_{p} ( i ) {T}_{g ( i , p )}^{∝ ( i , g ( i , p ) )} {e}^{\frac{{\sum }_{p}^{K} {t}_{p} {D}_{p} ( i ) {T}_{g ( i , p )}^{∝ ( i , g ( i , p ) )} - {D}_{o , r} ( i )}{c} + {S}_{r}}}{c} \\ \frac{\partial {f}_{organ}}{\partial {T}_{j}} = {\sum }_{p}^{K} {\sum }_{i}^{N} I ( p , i , j ) \frac{∝ ( i , g ( i , p ) ) {D}_{p} ( i ) {T}_{g ( i , p )}^{∝ ( i , g ( i , p ) ) - 1} {e}^{\frac{{\sum }_{p}^{K} {t}_{p} {D}_{p} ( i ) {T}_{g ( i , p )}^{∝ ( i , g ( i , p ) )} - {D}_{o , r} ( i )}{c} + {S}_{r}}}{c} \qquad ( Equation 20 )$$

Equation 20 provided partial derivatives with respect to the dwell time tᵢ and the transmission rate Tᵢ. In Equation 20, I(p,i,j) was an indicator function that defined whether, at dwell location p, the i^{th} pixel would be affected by the j^{th} transmission rate.

The cost function was then alternately minimized with respect to one variable (while fixing the other), using the following:

t^{k}=argmin_{S}_{ₜ}f(t,T^{k}), T^{k}=argmin_{S}_{_{T}}f(t^{k+1},T) (Equation 21)

where Sₜ={t|t [symbol] } and S_{T}={T|0.25 [symbol] T [symbol] 1}.

For each sub-problem, the gradient descent with back-tracking line search was used. Upon convergence, the constraints for the OARs were checked. If the constraints were not met, the cost function was automatically modified by changing sᵣ such that the OARs were favored. The compare the disclosed patient-specific tandem applicator method with the existing HDR method, the same optimization model, as described above, was used with the exception of fixing the transmission rates as a constant (T=1.0).

### Dose Constraints and Parameters

For both the experiments performed on the 2D phantom and the 2D patient data, the dose constraints outlined in Table 3 below were assigned to the CTV, bladder, rectum, and sigmoid. More specifically, Table 3 provided dose constraints as prescribed by the patient's physician. For the experiments performed on the 3D patient data, the dose constraint for the CTV was 560 cGy. The dose constraints for the bladder, rectum, and sigmoid were the same as those outlined in Table 3 for the 2D phantom and the 2D patient data.

For the patient data experiments, clinically treated cervical HDR patient data were used. A radioactive Ir-192 source was utilized, and data was collected for the twelve dwell positions of the Ir-192 source that were monitored.

TABLE 3
Dose Constraints Assigned to Structures in the 2D Phantom and 2D
Patient Cases

| Structure | Dose Constraint |
|---|---|
| CTV | ≥550 cGy |
| Bladder | ≤460 cGy |
| Rectum | ≤420 cGy |
| Sigmoid | ≤420 cGy |

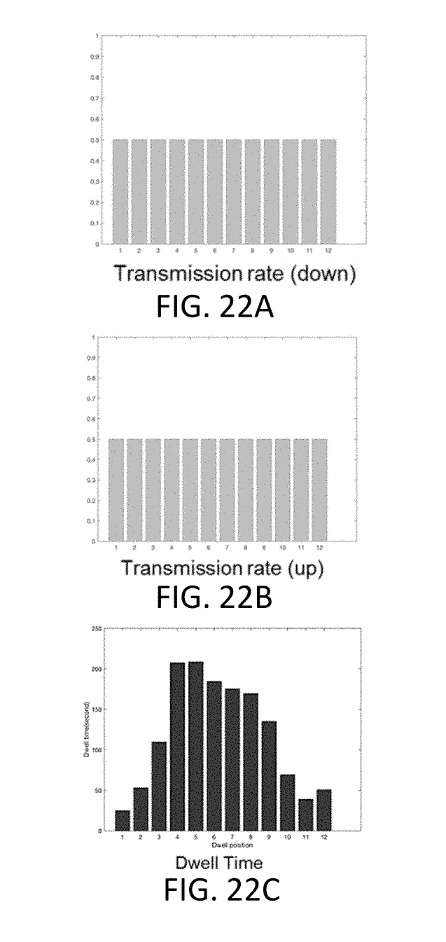

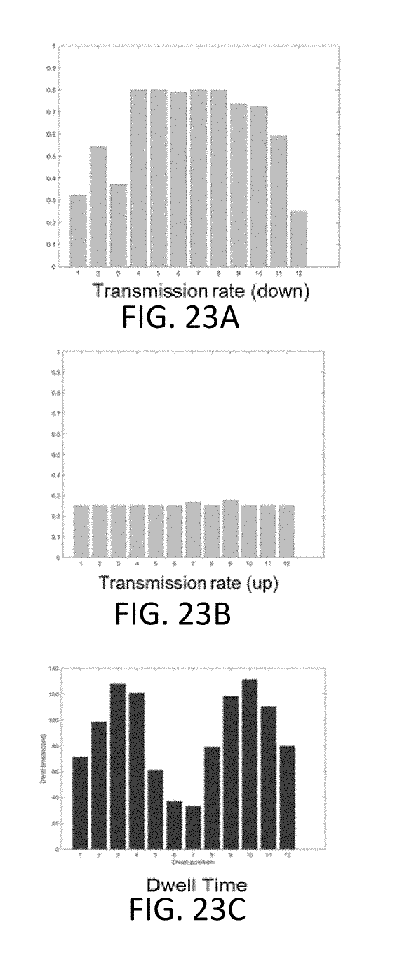

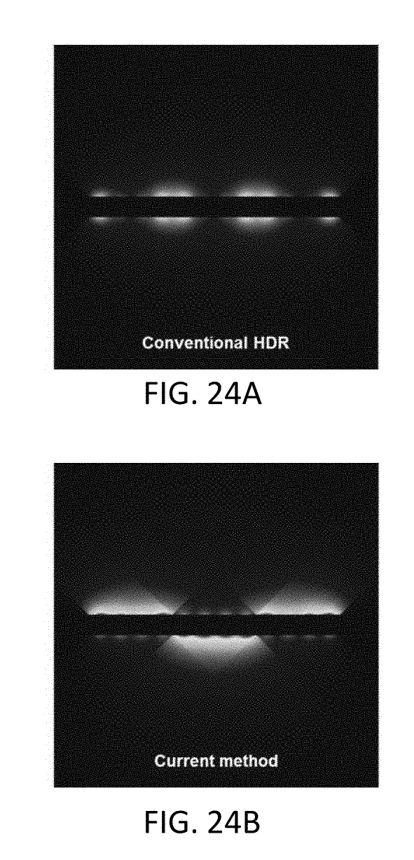

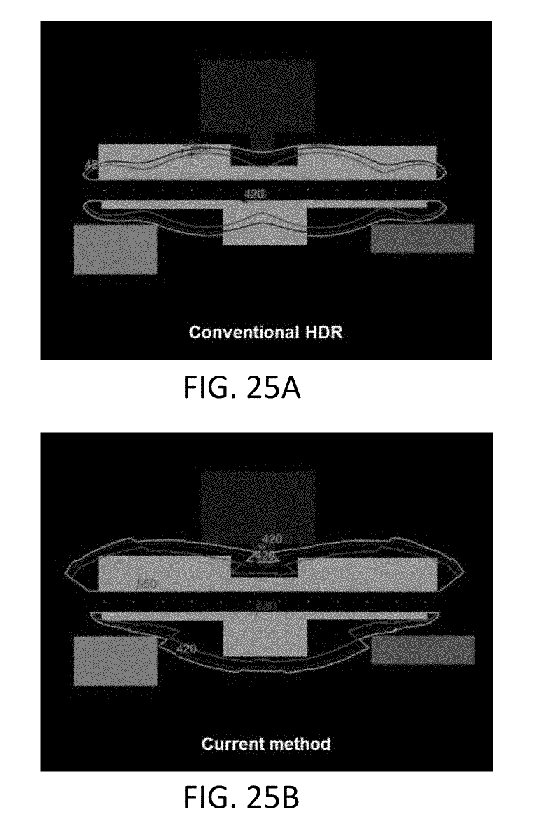

A configuration was calculated for an existing HDR method, as shown in FIGS. 22A, 22B, and 22C, to compare the existing HDR method to the patient-specific tandem applicator method. The calculated configuration for the patient-specific tandem applicator method using the HDR inverse planning optimization model is shown in FIGS. 23A-23C. Transmission rates and dwell times were calculated for each of twelve dwell positions. Similarly, dose distributions were calculated for both the existing HDR method and the patient-specific tandem applicator method described above. FIG. 24A illustrates the dose distribution for the existing HDR method, and FIG. 24B illustrates the dose distribution for the patient-specific tandem applicator method. The observable differences in the intensity profiles of the existing case (shown in FIG. 24A) and the patient-specific tandem applicator case (shown in FIG. 24B) demonstrated the directional treatment capabilities of the patient-specific tandem applicator as described herein. FIGS. 25A and 25B illustrate dose constraint isodose lines for the existing HDR method (shown in FIG. 25A) and for the patient-specific tandem applicator method (shown in FIG. 25B). In FIGS. 25A and 25B, the isodose lines are shown in red for 550 cGy, blue for 480 cGy, and green for 420 cGy. As illustrated in FIG. 25A and as summarized in Table 4 below, for the existing HDR method, 58.32% of the CTV was covered by the prescribed dose. In contrast, the directional profile of the disclosed patient-specific tandem applicator design allowed for a more conformal dose profile, resulting in 99.18% of the CTV being covered by the prescribed dose. As seen in FIGS. 25A and 25B, the OAR dose constraints were also satisfied for both the existing HDR method and the patient-specific tandem applicator method.

TABLE 4
2D Phantom Criterions checking

| | Bladder | Rectum | Sigmoid | CTV |
|---|---|---|---|---|
| Existing HDR | 100% | 100% | 100% | 58.32% |
| Patient-Specific | 100% | 100% | 100% | 99.18% |
| Tandem Applicator | | | | |
| Method | | | | |

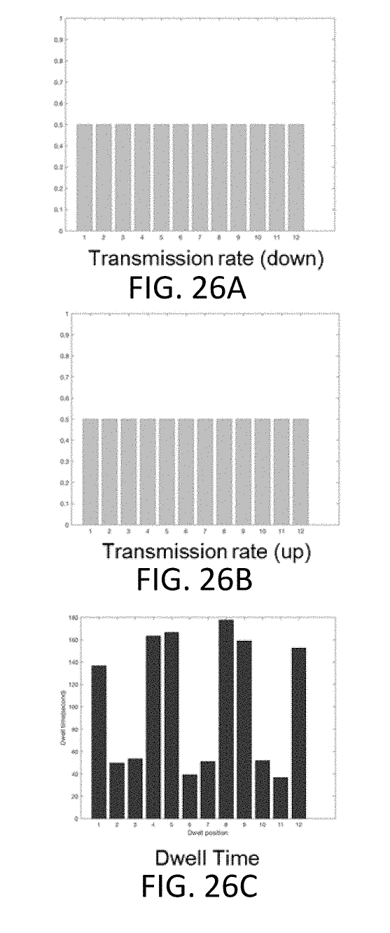

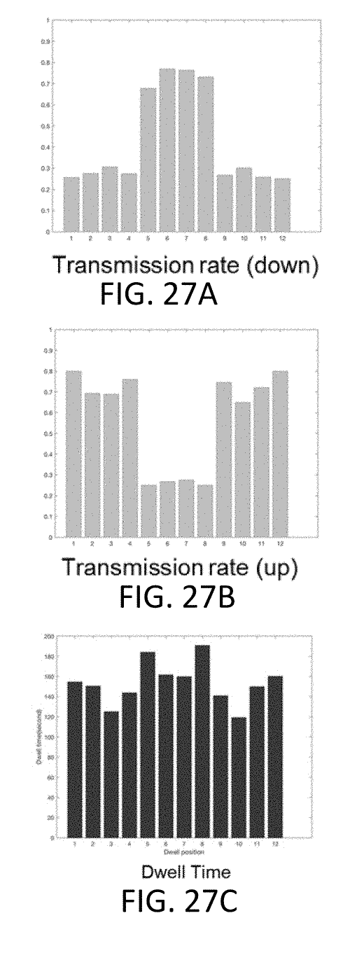

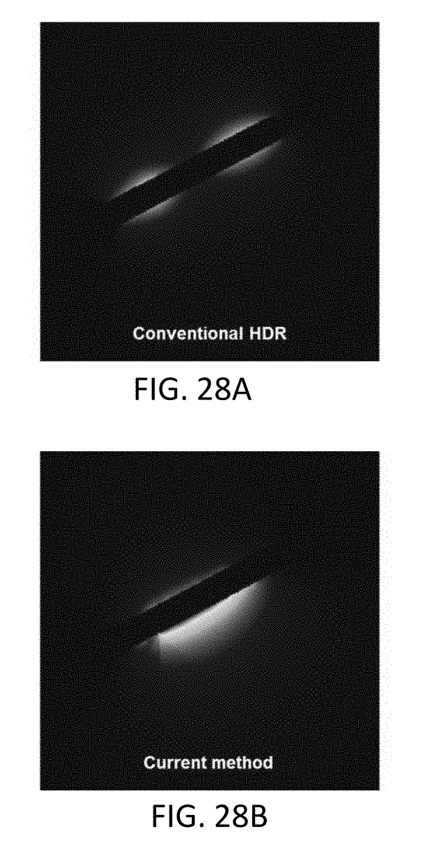

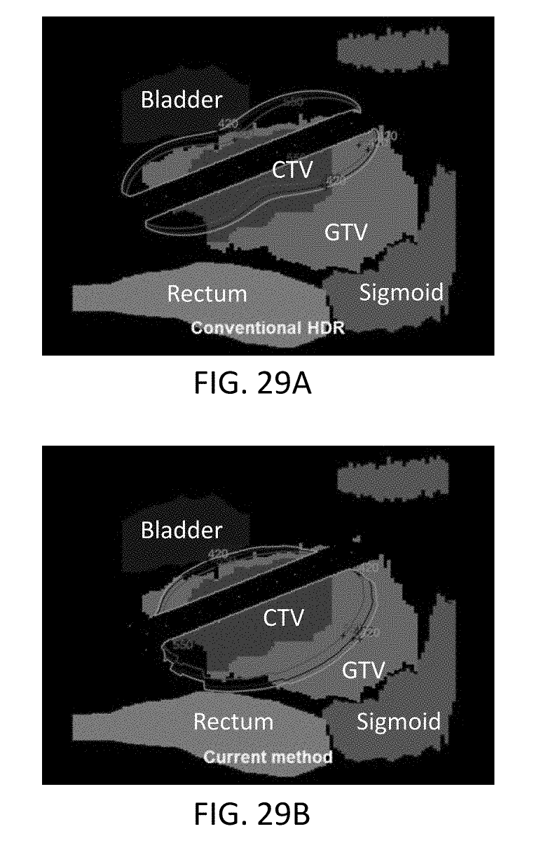

The advantages offered by the disclosed patient-specific tandem applicator design were demonstrated in the most realistic 2D patient case as well. A configuration was calculated using the 2D patient model for an existing HDR method, as shown in FIGS. 26A-26C and for the patient-specific tandem applicator method, as illustrated in FIGS. 27A-27CC. Similar to the 2D phantom results, FIGS. 26A-26C and 27A-27C provided calculations for transmission rates and dwell times at twelve different dwell positions. The dwell time for the 2D patient data experiments using patient-specific tandem applicator method was 23.78 minutes, which is comparable to existing IMRT treatments. FIGS. 28A and 28B illustrate the calculated dose distributions for the existing HDR method (shown in FIG. 28A) and the disclosed patient-specific tandem applicator method (shown in FIG. 28B). As seen in FIG. 28B, the directionality of the intensity profile was evident in the patient-specific tandem applicator case in comparison to the intensity profile of the existing HDR case shown in FIG. 28A. This directionality of the intensity profile as shown in FIG. 28B, resulted in better coverage of the CTV without sacrificing OAR sparing. FIGS. 29A and 29B illustrate dose constraint isodose lines for the existing HDR method (shown in FIG. 29A) and the patient-specific tandem applicator method (shown in FIG. 29B), with isodose lines shown in red for 550cGy, blue for 480 cGy, and green for 420 cGy. As illustrated in Table 5 below and in FIG. 29A 56.21% of the CTV was covered by the prescribed dose using the existing HDR method. In contrast, the directional profile of the patient-specific tandem applicator case resulted in a more conformal dose profile, with 99.92% of the CTV being covered by the prescribed dose without exceeding any OAR dose constraints, as shown in Table 5 and in FIG. 29B.

TABLE 5
2D Patient Data Criterions checking

| | Bladder | Rectum | Sigmoid | CTV |
|---|---|---|---|---|
| Existing HDR | 100% | 100% | 100% | 56.21% |
| Patient-Specific | 100% | 100% | 100% | 99.92% |
| Tandem Applicator | | | | |
| Method | | | | |

The comparison of treatments administered using the existing HDR method and the patient-specific tandem applicator method was repeated using 3D patient data. The 3D patient data included dimensions of 332×502×118 cm³ and had an image resolution of 0.29×0.29×0.9 cm³. Experiments on the 3D patient data were implemented using CUDA C++ to enable parallel computation. The computation time was under one minute using a system with the following specifications: a CPU of Intel i7-6700K 4.00 GHz, a GPU of NVidia GTX 1080, and a memory of 32 GB DDR4 3200 MHz.

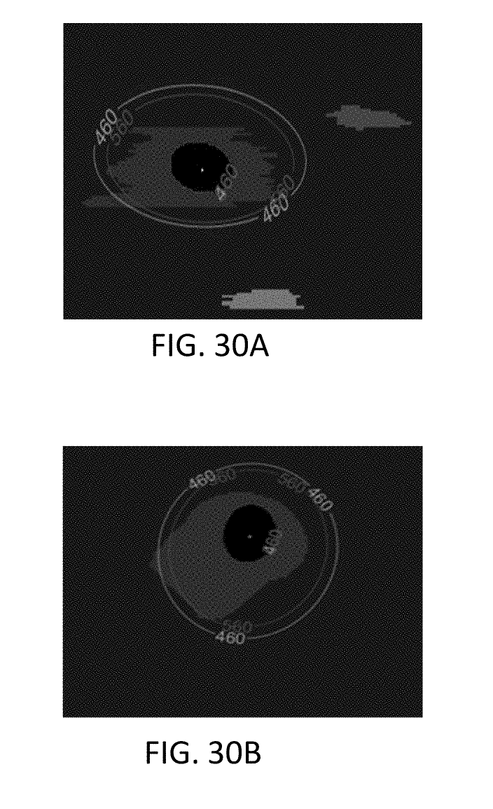

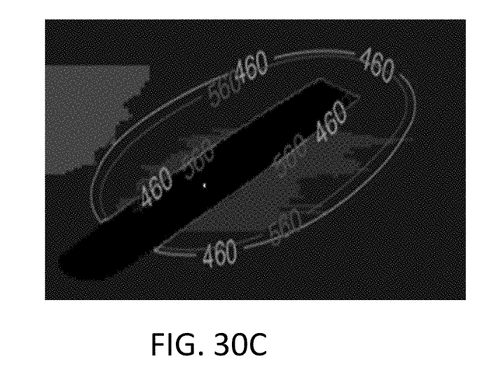

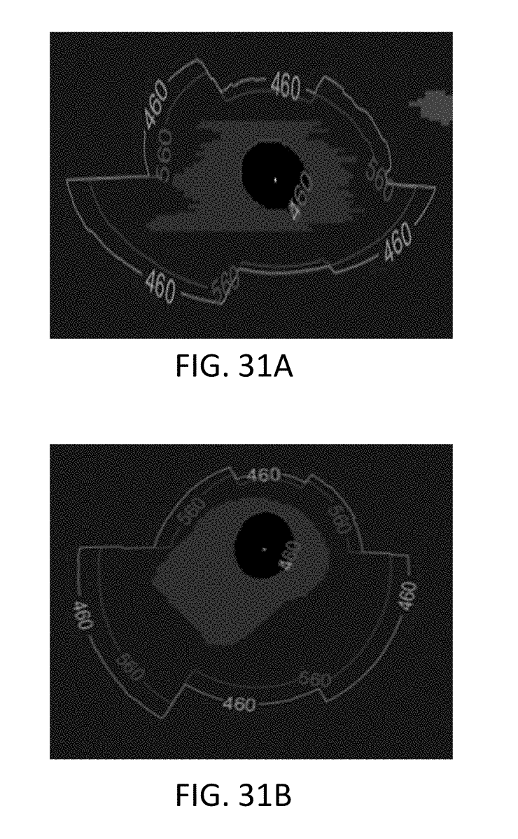

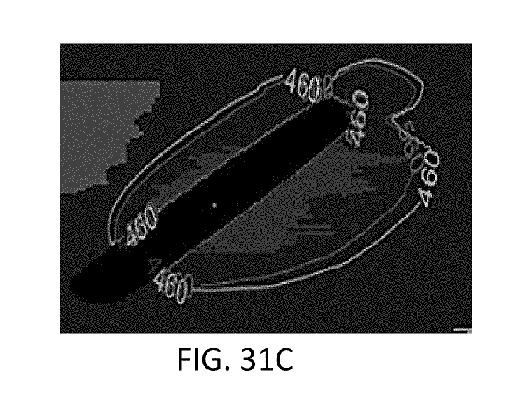

The patient-specific tandem applicator design yielded benefits when applied to the 3D patient data. FIGS. 30A, 30B, and 30C summarize dose constraint isodose lines for the existing HDR method, and FIGS. 31A, 31B, and 31C illustrate the corresponding dose constraint isodose lines for the disclosed patient-specific tandem applicator design. More specifically, FIGS. 30A, 30B, 31A, and 31B illustrate axial distributions at two slices. FIGS. 30C and 31C illustrate the distribution along the tandem axis for a single slice. Isodose lines are shown in red for 560 cGy, green for 460 cGy, and blue for 420 cGy. As illustrated in FIGS. 30A, 30B, and 30C and as illustrated in FIGS. 31A, 31B, and 31C, the directionally-modulated dose distribution achieved by patient-specific tandem applicator design improved coverage of the CTV from 90.02% using the existing HDR method to 99.97% using the patient-specific tandem applicator method (in the disclosed case). The directional dose profile in the disclosed case allowed for the coverage of extended portions of the tumor without compromising coverage of the tumor and OARs at emission angles, which was not achieved using the existing HDR method. The total treatment time for the patient using the patient-specific tandem applicator method was approximately 24 minutes, which was comparable to treatment time of existing IMRT treatments.

The results of these experiments validated the patient-specific tandem applicator design and treatment method. The patient-specific tandem applicator design improved dose coverage of the CTV by 9-44% without compromising the surrounding OARs. The patient-specific tandem applicator design yielded benefits when applied to the 2D patient case by covering 99.92% of the CTV in comparison to the existing HDR method only covering 56.21% of the CTV. For both the 2D phantom case and the 2D patient case, the complete tumor coverage was achieved while simultaneously satisfying the OAR constraints. The patient-specific tandem applicator method significantly improved the coverage by approximately 70% in the 2D phantom case and 78% in the 2D patient case. The patient-specific tandem applicator design also yielded benefits when applied to 3D patient data. The patient-specific tandem applicator design improved coverage of the CTV with respect to the existing HDR method without exceeding any OAR dose constraints when applied to the 2D phantom, the 2D patient data, or the 3D patient data.

What is claimed is:

1. A patient-specific intensity-modulated high dose rate (HDR) brachytherapy applicator for administering an HDR brachytherapy treatment to a patient, the applicator comprising a plurality of shielding segments distributed along a central longitudinal axis, each shielding segment corresponding to one dwell position and comprising a shielding wall, each shielding wall comprising a plurality of equiangular shielding sections of varying thickness distributed circumferentially about the central longitudinal axis, each equilangular shielding section comprising a shielding thickness, wherein each shield thickness of each equiangular shielding section at each shielding segment is configured to transmit radiation from an HDR source positioned within each shielding segment into the patient at a predetermined dose rate distribution to administer the HDR brachytherapy treatment.

2. The applicator of claim 1, wherein each shield thickness of each equiangular shielding section is independently determined using a computer-implemented inverse planning optimization model configured to determine each shield thickness based on a patient-specific radiation treatment plan.

3. The applicator of claim 1, wherein each shielding segment comprises from about 2 to about 10 equiangular shielding sections.

4. The applicator of claim 3, wherein each shielding segment comprises about 6 equiangular shielding sections.

5. The applicator of claim 1, wherein the applicator comprises tungsten metal formed using a 3D printing device.

6. A computer-implemented method for designing a patient-specific intensity-modulated high dose rate (HDR) brachytherapy applicator for administering an HDR brachytherapy treatment to a patient, the applicator comprising a plurality of shielding segments distributed along a central longitudinal axis, each shielding segment comprising a plurality of equiangular shielding sections distributed circumferentially about the central longitudinal axis, the method implemented using at least one processor in communication with at least one memory, the method comprising:
receiving, by a computing device, a radiation treatment plan for administering the HDR brachytherapy treatment, the radiation treatment plan comprising a prescribed radiation dosage to be delivered to a region of interest and patient anatomical data representative of the region of interest to be treated;
determining, by the computing device, an optimal shielding thickness profile and a plurality of optimal dwell times using an inverse planning optimization model constrained by the radiation treatment plan, each optimal dwell time corresponding to one dwell position, each dwell position corresponding to one shielding segment, and the optimal thickness profile comprising a plurality of shield thicknesses, each shield thickness corresponding to one equiangular shielding section of one shielding segment;
generating a dwell position-dependent shielding thickness profile comprising the positions of the plurality of the shielding segments and each shield thickness of each equiangular shielding section at each shielding segment; and
transmitting, by the computing device, design instructions to a three dimensional (3D) printer for fabrication of the applicator, wherein the design instructions include at least the dwell position-dependent shielding thickness profile.

7. The computer-implemented method of claim 6, wherein determining the optimal shielding thickness profile and the plurality of optimal dwell times using the inverse planning optimization model constrained by the radiation treatment plan further comprises:
calculating, by the computing device, a plurality of radiation dose rate maps and a plurality of transmission rate maps, each radiation dose rate map and each transmission rate map corresponding to one dwell position of the plurality of dwell positions;
calculating, by the computing device, a radiation dose distribution based on the plurality of radiation dose rate maps, the plurality of transmission rate maps, and the plurality of dwell times, the radiation dose distribution comprising a spatial map of a cumulative amount of radiation delivered from a HDR source positioned at each dwell position for each corresponding dwell time;
minimizing a cost function by alternately varying the plurality of dwell times with the plurality of transmission rate maps held constant and varying the plurality of transmission rate maps with the plurality of dwell times held constant; and
calculating the optimal shielding thickness profile and the plurality of optimal dwell times based on the plurality of transmission rate maps and the plurality of dwell times determined to minimize the cost function.

8. The computer-implemented method of claim 7, wherein minimizing the cost function further comprises alternately minimizing the cost function by utilizing a gradient descent with back-tracking line search.

9. The computer-implemented method of claim 6, further comprising:
generating a dwell position-dependent dwell time schedule for the applicator comprising the plurality of dwell positions and a corresponding plurality of optimal dwell times; and
transmit the dwell position-dependent dwell time schedule to a treatment device for administering the HDR brachytherapy treatment to the patient using the applicator.

10. The computer-implemented method of claim 6, wherein each shielding segment comprises six equiangular shielding sections.

11. The computer-implemented method of claim 6, wherein the 3D printing device fabricates the applicator from tungsten metal.

12. A computing device for designing a patient-specific intensity-modulated high dose rate (HDR) brachytherapy applicator for administering an HDR brachytherapy treatment to a patient, the applicator comprising a plurality of shielding segments distributed along a central longitudinal axis, each shielding segment comprising a plurality of equiangular shielding sections distributed circumferentially about the central longitudinal axis, the computing device including at least one processor in communication with at least one memory device, the at least one processor programmed to:
receive a radiation treatment plan for administering the HDR brachytherapy treatment, the radiation treatment plan comprising a prescribed radiation dosage to be delivered to a region of interest and patient anatomical data representative of the region of interest to be treated;
determine an optimal shielding thickness profile and a plurality of optimal dwell times using an inverse planning optimization model constrained by the radiation treatment plan, each optimal dwell time corresponding to one dwell position, each dwell position corresponding to one shielding segment, and the optimal thickness profile comprising a plurality of shield thicknesses, each shield thickness corresponding to one equiangular shielding section of one shielding segment;
generate a dwell position-dependent shielding thickness profile comprising the positions of the plurality of the shielding segments and each shield thickness of each equiangular shielding section at each shielding segment; and
transmit design instructions to a three dimensional (3D) printer for fabrication of the applicator, wherein the design instructions include at least the dwell position-dependent thickness profile.

13. The computing device of claim 12, wherein the at least one processor is further programmed to determine the optimal shielding thickness profile and the plurality of optimal dwell times using an inverse planning optimization model constrained by the radiation treatment plan by:
calculating, by the computing device, a plurality of radiation dose rate maps and a plurality of transmission rate maps, each radiation dose rate map and each transmission rate map corresponding to one dwell position of the plurality of dwell positions;
calculating, by the computing device, a radiation dose distribution based on the plurality of radiation dose rate maps, the plurality of transmission rate maps, and the plurality of dwell times, the radiation dose distribution comprising a spatial map of a cumulative amount of radiation delivered from a HDR source positioned at each dwell position for each corresponding dwell time;
minimizing a cost function by alternately varying the plurality of dwell times with the plurality of transmission rate maps held constant and varying the plurality of transmission rate maps with the plurality of dwell times held constant; and
calculating the optimal shielding thickness profile and the plurality of optimal dwell times based on the plurality of transmission rate maps and the plurality of dwell times determined to minimize the cost function.

14. The computing device of claim 13, wherein the at least one processor is further programmed to minimize the cost function using a gradient descent with back-tracking line search.

15. The computing device of claim 12, wherein the at least one processor is further programmed to:
generate a dwell position-dependent dwell time schedule for the applicator comprising the plurality of dwell positions and a corresponding plurality of optimal dwell times; and
transmit the dwell position-dependent dwell time schedule to a treatment device for administering the HDR brachytherapy treatment to the patient using the applicator.

16. The computing device of claim 12, wherein each shielding segment comprises six equiangular shielding sections comprising tungsten.

17. The computing device of claim 16, wherein each shield thickness ranges from about 0.12 cm to about 0.48 cm.

18. A high-dose radiation (HDR) modulating system configured to improve target coverage of tumor volume during an HDR treatment, the HDR modulating system including:
a patient-specific intensity-modulated high dose rate (HDR) brachytherapy applicator comprising a plurality of shielding segments distributed along a central longitudinal axis, each shielding segment comprising a plurality of equiangular shielding sections distributed circumferentially about the central longitudinal axis, the plurality of shielding segments defining a central lumen extending along the central longitudinal axis, each shielding segment further defining a dwell position within the central lumen; and
an HDR source movably insertable into the central lumen during an HDR treatment, the HDR source configured to reside at each dwell position within each shielding segment for a corresponding dwell time, wherein each corresponding dwell time is based on a radiation therapy plan;
wherein each equiangular shielding section at each shielding segment comprises a shield thickness configured to transmit radiation from the HDR source residing at each dwell position at a predetermined dose rate distribution.

19. The system of claim 18, wherein the exterior surface of the applicator further comprises an indicator configured to orient the applicator relative to a region of interest to be treated, wherein the indicator is configured to be visible on a three-dimensional imaging system.

20. The system of claim 18, wherein each equiangular shielding section comprises tungsten and each shield thickness ranges from about 0.12 cm to about 0.48 cm.
